(19) United States
(12) Patent Application Publication
(10) Pub. No.: US 2005/0043263 A1
(43) Pub. Date: Feb. 24, 2005

(54) Use of double-stranded ribonucleic acid for inducing cell lysis

(75) Inventors: Klaus Giese, Berlin (DE); Joerg Kaufmann, Berlin (DE)

Correspondence Address: HELLER EHRMAN WHITE & MCAULIFFE LLP, 1666 K STREET,NW, SUITE 300, WASHINGTON, DC, 20006, US

(73) Assignee: Atugen AG, Berlin (DE)

(21) Appl. No.: 10/876,779

(22) Filed: Jun. 28, 2004

Related U.S. Application Data
(60) Provisional application No. 60/520,671, filed on Nov. 18, 2003.

(30) Foreign Application Priority Data
Jun. 27, 2003 (DE) 03014766.4

(51) Int. Cl. A61K 48/00

(52) U.S. Cl. 514/44

(57) ABSTRACT

The present invention is related to the use of a ribonucleic acid for the manufacture of a medicament, wherein the ribonucleic acid comprises a double-stranded structure and the double-stranded structure comprises a first and a second strand, wherein the first strand comprises a first stretch of contiguous nucleotides and the second strand comprises a second stretch of contiguous nucleotides, wherein the first stretch is not complementary to a nucleic acid, preferably a mRNA, of a cell of an organism to be treated with said medicament and/or wherein the second stretch is different from a nucleic acid, preferably a mRNA of a cell of an organism to be treated with said medicament.

This application claims priority to application Ser. No. 60/520,671, filed Nov. 18, 2003 and European Application No. 03014766.4, filed Jun. 27, 2003, the contents of each of which are incorporated herein by reference in their entireties.

### BACKGROUND OF THE INVENTION

The present invention is related to a double-stranded nucleic acid comprising a first strand and a second strand which are essentially complementary to each other and the use thereof as a medicament.

Undesired cell growth in general and tumors in particular, are in many cases the result of a loss of function. This uncontrolled cell growth is the target for various approaches chosen for the treatment of tumors. Such approaches comprise, among others, the use of compounds which allow the elimination of over-expressed factors, which is in some cases a direct consequence of the loss of function of tumor suppressors. Suitable means for that purpose are, among others, small molecules which are screened against said molecule or the removal of this kind of factor by, among others, antibodies from the site of action. In any of these cases, however, the treatment must be an on-going one as otherwise the imbalanced factor would again be over-expressed or over-present once the respective means is no longer provided to the organism suffering from that disease. Other approaches make use of physiological phenomena typically observed in connection with tumors, such as angiogenesis. An approach which seems to be successful, is the use of antibodies against VEGF so as to inhibit the supply of nutrients to the tumor. However, as angiogenesis is a crucial biological phenomenon, side effects are highly relevant and need careful attention.

A further strategy pursued in the treatment of tumors and tumor-related diseases is the use of oncolytic viruses. Such viruses, among others, adenoviruses, are used to infect a tumor cell which upon infection undergoes apoptosis thus lysing the tumor as such. However, virus-mediated oncolysis typically requires a certain genetic background of the cells and tumors, respectively, to be treated so as to provide for a tumor-selective lysis of the cells.

Small interfering RNA, also referred to as siRNA, has attracted considerable attention as a means for sequence specific inhibition of the expression of a target gene. siRNA is mediating a phenomenon which is called RNA-mediated interference (RNAi) which is a post-transcriptional gene silencing mechanism initiated by double-stranded RNA (dsRNA) homologous in sequence to the silenced gene (Fire (1999), Trends Genet 15, 358-63, Tuschl, et al. (1999), Genes Dev 13, 3191-7, Waterhouse, et al. (2001), Nature 411, 834-42, Elbashir, et al. (2001), Nature 411, 494-8, for review see Sharp (2001), Genes Dev 15, 485-90, Barstead (2001), Curr Opin Chem Biol 5, 63-6). RNAi has been used extensively to determine gene function in a number of organisms, including plants (Baulcombe (1999), Curr Opin Plant Biol 2, 109-13), nematodes (Montgomery, et al. (1998), Proc Natl Acad Sci USA 95, 15502-7), Drosophila (Kennerdell, et al. (1998), Cell 95, 1017-26, Kennerdell, et al. (2000), Nat Biotechnol 18, 896-8). In the nematode C. elegans about one third of the genome has already been subjected to functional analysis by RNAi (Kim (2001), Curr Biol 11, R85-7, Maeda, et al. (2001), Curr Biol 11, 171-6).

Until recently RNAi was not generally applicable in mammalian cells, with the exception of early mouse development (Wianny, et al. (2000), Nat Cell Biol 2, 70-5). The discovery that transfection of duplexes of 21 nt long ribooligonucleotides into mammalian cells interfered with gene expression and did not induce a sequence independent interferon-driven anti-viral response usually obtained with long dsRNA led to new potential applications in differentiated mammalian cells (Elbashir et al. (2001), Nature 411, 494-8). Interestingly, these small interfering RNAs (siRNAs) resemble the processing products from long dsRNAs suggesting a potential bypassing mechanism in differentiated mammalian cells. The Dicer complex, a member of the RNAse III family, necessary for the initial dsRNA processing has been identified (Bernstein, et al. (2001), Nature 409, 363-6, Billy, et al. (2001), Proc Natl Acad Sci USA 98, 14428-33). One of the problems previously encountered when using unmodified ribooligonucleotides was the rapid degradation in cells or even in serum-containing medium (Wickstrom (1986), J Biochem Biophys Methods 13, 97-102, Cazenave, et al. (1987), Nucleic Acids Res 15, 10507-21). It will depend on the particular gene function and assay systems used whether the respective knock-down induced by transfected siRNA will be maintained long enough to achieve a phenotypic change.

The main characteristic of siRNA as used to date is that one strand of the double-stranded structure is complementary to the target nucleic acid sequence whereas the other is, due to the base pairing mechanism underlying the double-stranded structure of siRNA, identical to the target nucleic acid. Any nucleotides or sequences attached to either side of any of the two strands forming the double-stranded structure of siRNA is added for purposes of cloning or stabilisation.

To date, siRNA does not seem to be a suitable means for the treatment of tumors and tumor diseases, respectively. Even if an imbalanced factor is degraded by means of siRNA, such degradation has to be sustained for a very long time as the cells forming the tumor are not eliminated by the siRNA. On the other hand, those forms of tumors which are not characterised by the loss of a control factor but by an aberrant but otherwise physiological factor, require a highly specific siRNA which addresses solely the aberrant form. However, for the design of such siRNA, it is necessary to know the kind of nucleic acid sequence which is responsible for the respective aberrant form of the imbalanced factor. Although it is to be acknowledged that for a certain group of tumors the same aberrations occur, a careful molecular biological characterisation of the tumors to be treated by siRNA would thus be required which is very time and money consuming. Additionally, given the high specificity of siRNA it seems questionable whether a siRNA species designed for the treatment of a distinct tumor of a certain group of patients would actually be suitable for the treatment of the same tumor of a different group of patients.

Therefore, the currently envisaged use of siRNA meets some concerns.

### SUMMARY OF THE INVENTION

It is, therefore, a problem underlying the present invention to provide means for the treatment of tumors and tumor-related diseases, and to provide respective compounds therefor.

In a first aspect the problem underlying the present invention is solved by the use of a nucleic acid, preferably a ribonucleic acid, for the manufacture of a medicament,
wherein the nucleic acid comprises a double-stranded structure and the double-stranded structure comprises a first and a second strand,
wherein the first strand comprises a first stretch of contiguous nucleotides and the second strand comprises a second stretch of contiguous nucleotides,
wherein the first stretch is not complementary to a target nucleic acid, preferably a mRNA, of a cell of an organism to be treated with said medicament and/or
wherein the second stretch is different from a target nucleic acid, preferably a mRNA of a cell of an organism to be treated with said medicament.

In an embodiment the target nucleic acid is any nucleic acid of a cell of an organism, preferably any mRNA of a cell of an organism.

In an embodiment the target nucleic acid is any element of the transcriptome, preferably all elements of the transcriptome of a cell of an organism.

In an embodiment the cell is a pathological cell which is preferably involved in the disease.

In an embodiment the first stretch is complementary to a nucleic acid, preferably a mRNA, of a non-pathological cell of the organism to be treated and/or
the second stretch is identical to a target nucleic acid, preferably a mRNA of a non-pathological cell of the organism to be treated.

In a preferred embodiment the target nucleic acid of the non-pathological cell differs from the target nucleic acid of the pathological cell at one or more nucleotide positions.

In an embodiment the medicament is for the treatment and/or the prevention of a disease, wherein such disease is preferably tumor or cancer.

In an embodiment the pathological cell is tumor suppressor-defective and the first stretch of contiguous nucleotides is complementary to the nucleic acid coding for the functional tumor suppressor and/or
the second stretch of contiguous nucleotides is identical to the nucleic acid coding for the functional tumor suppressor.

In a preferred embodiment the first stretch is complementary to the nucleic acid coding for the functional tumor suppressor for which the pathological cell is tumor suppressor-defective, and
the second stretch is identical to the nucleic acid coding for the functional tumor suppressor for which the pathological cell is tumor suppressor-defective.

In an embodiment the pathological cell is lacking a gene of the functional tumor suppressor or a transcript thereof.

In an embodiment the pathological cell is lacking a gene or a transcript thereof providing for a functionally active tumor suppressor

In a preferred embodiment the gene or the transcript thereof comprises one or several mutations, wherein such mutation is preferably selected from the group comprising point mutations and deletion mutations, wherein such mutation(s) preferably results in a functionally inactive tumor suppressor.

In a second aspect the problem underlying the present invention is solved by the use of a nucleic acid, preferably a ribonucleic acid for the manufacture of a medicament, wherein the nucleic acid comprises a double-stranded structure and the double-stranded structure comprises a first strand and a second strand,
wherein the first strand comprises a first stretch of contiguous nucleotides and the second strand comprises a second stretch of contiguous nucleotides,
wherein the nucleic acid or part or a strand thereof is RNA interference response-negative.

In an embodiment of the second aspect the nucleic acid is RNA interference response-negative in pathological cells of the organism to be treated with the medicament.

In an embodiment of the second aspect the nucleic acid is RNA interference response-positive in non-pathological cells of the organism to be treated with the medicament.

In an embodiment of the first and the second aspect the nucleic acid induces a stress response, preferably apoptosis and/or inhibition of proliferation of the pathological cells.

In a third aspect the problem underlying the present invention is solved by a pharmaceutical composition comprising a nucleic acid, preferably a ribonucleic acid, wherein the nucleic acid comprises a double-stranded structure and the double-stranded structure comprises a first strand and a second strand,
wherein the first strand comprises a first stretch of contiguous nucleotides and the second strand comprises a second stretch of contiguous nucleotides,
wherein the first stretch is not complementary to a target nucleic acid, preferably a mRNA, of a cell of an organism to be treated with said medicament and/or
wherein the second stretch is different from a target nucleic acid, preferably a mRNA of a cell of an organism to be treated with said medicament;
and preferably a pharmaceutically acceptable carrier.

In an embodiment of the third aspect the target nucleic acid is any nucleic acid of a cell of an organism to be treated using said pharmaceutical composition, preferably any mRNA of a cell of such organism.

In an embodiment of the third aspect the target nucleic acid is any element of the transcriptome, preferably all elements of the transcriptome of a cell of an organism to be treated with said pharmaceutical composition.

In an embodiment of the third aspect the cell is a pathological cell which is preferably involved in the disease which is to be treated and/or prevented by the pharmaceutical composition.

In an embodiment of the third aspect the nucleic acid is as defined in connection with the first aspect of this invention.

In an embodiment of the third aspect the pharmaceutical composition further comprises at least one lipid, preferably a cationic lipid.

In a preferred embodiment of the third aspect the lipid is beta-arginyl-2,3-diaminopropionic acid-N-palmityl-N-oleyl-amide trihydrochloride.

In an embodiment of the third aspect the pharmaceutical composition further comprises a helper lipid.

In a preferred embodiment of the third aspect the helper lipid is diphytanoylphosphatidylethanolamine.

In a fourth aspect the problem underlying the present invention is solved by a method for the treatment of a patient in need of such treatment, comprising the step of administering a pharmaceutical composition according to the third aspect and/or a nucleic acid as described in connection with the first aspect of this invention, preferably for the treatment of cancer and/or tumor.

In an embodiment of the fourth aspect the patient exhibits cells, preferably pathological cells, as defined in any of the preceding claims.

### BRIEF DESCRIPTION OF THE DRAWINGS

FIG. 1 shows the principle reactions of small RNA duplexes with FIG. 1A illustrating the principle mechanism underlying the present invention, whereas FIG. 1B illustrates the mode of action of siRNA;

FIG. 2A shows various siRNA construct as applicable in the present invention;

FIG. 2B shows the result of an immunoblot of cell lysates using either siRNA or antisense molecules designed against PTEN;

FIG. 2C shows the result of an immunoblot of cell lysates using either siRNA or antisense molecules designed against PTEN, wherein apart from p100α, PTEN and pAkt also pFKHR is used as read-out;

FIG. 3A shows a diagram depicting the increase and decrease of the number of probe sets of an affymatrix experiment using different antisense constructs and siRNA constructs, respectively, after 12 and 24 hours;

FIG. 3B shows the results of an affymatrix experiment using different antisense and RNA constructs for PTEN mRNA knock-down;

FIG. 4A shows a diagram depicting the volume of PC3 tumours upon treatment with various siRNA expression constructs;

FIG. 4B shows a diagram depicting the volume of HeLa tumours upon treatment with various siRNA expression constructs;

FIG. 5A shows PC-3 cells in a cell proliferation assay, wherein the cells were subjected to transfection using different RNA molecules, after 72 h;

FIG. 5B shows PC-3 cells in a cell proliferation assay, wherein the cells were subjected to transfection using different RNA molecules, after 160 h;

FIG. 6 shows the Western Blot analysis of cell lysates after treatment of HeLa cells using different double-stranded RNA molecules;

FIG. 7 shows a diagram depicting the absolute tumor volume as a function of time expressed as days post cell challenge using different formulations;

FIG. 8 shows a diagram depicting the tumor volume as a function of days post cell challenge upon administration of PBS and different concentrations of a lipid formulation containing a double-stranded ribonucleic acid; and

FIG. 9 shows a diagram depicting the tumor volume as a function of days post cell challenge upon administration of PBS and different concentrations of a lipid formulation containing a double-stranded ribonucleic acid.

### DETAILED DESCRIPTION

The present inventors have surprisingly found that it is possible to use double-stranded nucleic acid for the treatment of various diseases including, but not limited to, tumor and tumor associated diseases. Preferably, the use of double-stranded nucleic acid according to the present invention results in a stress response. Such stress response leads to any of the following phenomena which are individually or in any combination also referred to as stress response herein, namely cell cycle arrest, growth inhibition, cell death, apoptosis, elimination of cells, preferably elimination of cell through or mediated by apoptosis, or the induction of any of these phenomena, wherein the cells are either in a causative manner or in a non-causative manner, i. e. directly or indirectly, involved in said diseases. Additionally, it is the current understanding of the present inventors that the stress response may either directly or indirectly involve, cause or be caused by an innate immune response and/or an antiviral response by or in a cell, tissue, organ or organism treated with the double-stranded nucleic acid. It is to be understood that the present invention can be practiced without knowing the mode of action of the double-stranded nucleic acid and without exactly knowing the mechanism(s) resulting in said stress response and more particularly in one or several aspects thereof such as apoptosis and cell lysis, respectively

Additionally, the present inventors have surprisingly found that the stress response can also be triggered in the presence of an RNA interference response. This can be achieved if the cell is confronted with an amount of RNAi molecules which is specific to a target nucleic acid and which can not be dealt with by the RNA interference machinery which results in an overflow of such machinery. Thus overflow of RNAi molecules channels the abundant or surplus RNAi molecules into a different pathway which results in the stress response described herein.

The double-stranded nucleic acid which is used according to the present invention and which is also referred to herein as the double-stranded nucleic acid according to the present invention, comprises a first strand and a second strand. The first strand comprises a first stretch of contiguous nucleotides and the second strand comprises a second stretch of contiguous nucleotides. Both strands are base pairing, preferably through Watson-Crick base pairing. More preferably, the base pairing is perfect, i.e. there is no mismatching between the nucleotides of the first stretch and the nucleotides on the second stretch.

The present inventors have also surprisingly found that by using this kind of double-stranded nucleic acid, the stress response as defined above can be caused, provided that the sequence of the first stretch or the first strand is not complementary to a nucleic acid, wherein the nucleic acid is preferably a target nucleic acid, more preferably such nucleic acid is contained in a transcription system such as a cell, tissue, organ or organism. This kind of nucleic acid will generally be referred to herein as target nucleic acid. Such lack of complementarity can basically be created under the following two scenarios. The first scenario is that there is no such target nucleic acid present in the transcription system, wherein such transcription system is preferably a cell, and wherein such target nucleic acid is more preferably not present in the transcriptome of the cell. This scenario is also referred to herein as lack of gene which results in the above described stress response. Under the lack of gene scenario, there is preferably no RNA interference response, however, there are embodiments where such RNA interference response may be present. An assay on how such stress response can be measured is provided in the examples. Under the second scenario, the target nucleic acid is basically present, however, the sequence of the first stretch and first strand, respectively, of the double-stranded nucleic aicd according to the present invention comprises one or several mismatches relative or compared to a target nucleic acid present in the cell, and more preferably relative to the transcriptome of the cell. In other words, by providing one or several mismatches in the first stretch and strand, respectively, which provides that the complementarity relative to any target nucleic acid as present in a transcription system such as a cell and preferably to the transcriptome of the cell is no longer given, the same effect is realized as under the first scenario. Because of this design, there is no interaction or relationship between the target nucleic acid and the first stretch and first strand, respectively, of the double-stranded nucleic acid according to the present invention in terms of complementarity, wherein the lack of this kind of interaction or relationship provides for the above described stress response and usually does not go along with the triggering of an RNA interference reaction as known in the art, or at least with the result thereof as observed under the condition of a match of the first stretch and second stretch, respectively, of the double-stranded nucleic acid according to the present invention and the target nucleic acid. There is therefore a gradual change or transition, when it comes to complementarity and mismatches as inherent to the first and the second scenario outlined herein and more particularly in this paragraph. The extent to which such mismatch(es) is/are required to trigger said stress response can be determined for each and any individual case by routine analysis using the assay described in the examples herein. In any case it is preferred that the first stretch and first strand, respectively, comprising mismatches are also not complementary in the sense provided above, to any other target nucleic acid of the transcription system, preferably the cell, or another target nucleic acid of the transcriptome of the cell.

The same considerations are applicable for the design of the second strand and the second stretch, respectively, wherein for this second stretch and second strand, respectively, the criterion is identity rather than complementarity, wherein in the preferred embodiment where the first stretch and the second stretch, and the first strand and the second strand, respectively, are perfectly matched, i. e. without any mismatch(es) between them, this will automatically be realized if the first strand and first stretch, respectively, is designed as outlined above.

Without wishing to be bound by any theory, it seems that the various proteins and factors, respectively, involved in RNA interference which is also referred to herein as RNA interference response, compare one strand, preferably the antisense strand which is preferably the first strand as used herein in connection with the double-stranded nucleic acid according to the present invention, to the target nucleic acid. In case there is a positive response in the meaning that the antisense strand provided by the double-stranded nucleic acid according to the present invention matches with the target nucleic acid such as the mRNA strand, a structure is produced which, either directly or indirectly, is recognized by nucleases involved in RNA interference (e.g. RISC complex) and allows for the degradation of the target nucleic acid. A double-stranded RNA which provides for this kind of reaction, such as siRNA, is also referred to herein as RNA interference response-positive. If, however, the respective factors cannot find any target nucleic acid which matches to one strand, preferably the antisense strand, of the siRNA, such signal or structure is not provided, or a different or additional signal is provided which, in the end, results in the stress reaction or stress response as defined above. A double-stranded nucleic acid which causes or triggers this kind of stress response, is also referred to as RNA interference response-negative, although it cannot be excluded that at least in some embodiments some RNA interference response can occur. Therefore, also double-stranded nucleic acid according to the present invention having mismatches in the broader sense between one of the strands of the double-stranded nucleic acid according to the present invention, preferably the antisense strand more preferably the first strand, and the target nucleic acid which is preferably each and any of the nucleic acids of the transcription system, more preferably any nucleic acid of the transcriptome of the transcription system such as a cell, provides for a signal which preferably results in apoptosis or cell death. However, the present inventors assume that further mechanisms beyond the RNA inference mechanism are involved in mediating the observed effect of the stress response upon the administration of a double-stranded nucleic acid according to the present invention, i.e. one not interacting with a target nucleic acid in the sense of triggering a RNA interference response. Further components involved in this stress response may be part of, e.g. the PKR pathway or any other interferon related pathways or any pro-apoptotic pathways.

According to the current understanding of the inventors, such a situation may preferably arise if a total of 12, 13, 14 contiguous nucleotides which are perfectly matching and are thus not interrupted by one or more mismatches, or less of the complementary strand or of the identical strand of the double-stranded ribonucleic acid according to the present invention do not match with or are not identical to the target nucleic acid. If preferably a total of about 15 or more nucleotides is either complementary or identical to the target nucleic acid, this will result in an RNA interference response and thus not in apoptosis. The latter is particularly applicable in case the second scenario is realized, i. e. that the target nucleic acid is not recognized because of the mismatches present in the first stretch and the first strand, respectively, and/or in the second stretch and the second strand, respectively. More preferably, this design is not to be taken into consideration if the target nucleic acid as such is not present in the cell and its transcriptome, respectively.

The present inventors have more particularly found that if the double-stranded nucleic acid according to the present invention is designed against or addresses a tumor suppressor and such tumor suppressor is not present in the tumor cells, this will result in a RNA interference response-negative reaction and thus in the stress response described above and in the end in apoptosis or cell death or growth inhibition.

However, the applicability of the double-stranded nucleic acid according to the present invention is not limited to tumors, tumor diseases, cancer and cancer diseases, wherein both malignant and benign tumors and cancers are comprised, but may also be applicable to other diseases. More preferably, the double-stranded nucleic acid according to the present invention may always be applied where a loss of function or loss of gene which results in such loss of function forms the basis of a disease or pathological condition, wherein the respective cell having or showing this loss of gene, preferably within an organism, is to be eliminated. In an embodiment the double-stranded nucleic acid according to the present invention which provides mismatches to any target nucleic acid of a transcription system, preferably provides mismatches when one strand thereof is base pairing with the target nucleic acid in a manner not to trigger the RNA interference response in this kind of cell, is preferably administered to the cell and the organism, respectively.

Still further diseases which may be treated by using the double-stranded nucleic acid according to the present invention, are those diseases where the disease involves, either directly or indirectly, a peptide or a protein which is encoded by a nucleic acid or a respective mRNA, wherein such nucleic acid or mRNA exhibits, compared to the wild type, one or more nucleotide exchanges. In other words, further diseases which can be treated according to the present invention are those diseases which exhibit one or several nucleotide polymorphism(s) (SNP(s)). Such nucleic acid or mRNA being in the diseased cell, tissue, organ and organism or any cell, tissue, organ and organism, respectively, having a predispostion to develop such disease and which is different from the nucleic acid or mRNA of the undiseased or healthy cell, tissue, organ and organism is then regarded as the target nucleic acid or as part of the nucleic acids of the transcriptions system, preferably as one of the nucleic acid of the transcriptome of the transcription system such as a cell. When designing the double-stranded nucleic acid according to the present invention this kind of target nucleic acid has also to be taken into consideration. Therefore, the double-stranded nucleic acid, more particularly the first stretch and first strand thereof which is the antisense strand for the target nucleic acid, may not be complementary to the nucleic acids of the transcription system and more particularly to the transcriptome of a cell such as the cell(s) involved either directly or indirectly in the disease, so as to allow the triggering or generation of the stress response as defined herein. In connection therewith it is to be noted that the respective double-stranded nucleic acid according to the present invention is preferably to encompass that part of the target nucleic acid which hosts the SNP or at least one thereof as otherwise a discrimination between those cells, tissues, organs or patients not having the SNP(s) would not be possible. In other words, the SNP(s) defines the nucleotide stretch on the target nucleic acid for which the first stretch and first strand of the double-stranded nucleic acid according to the present invention is not complementary as defined herein, wherein such lack of complementary is such as to trigger the stress response as defined herein. It is to be acknowledged that, of course, preferably, the double stranded nucleic acid according to the present invention is also not complementary to any other nucleic acid of the transcription system as taught herein.

It is within the present invention that treatment as used herein also comprises prevention. In one embodiment, particularly in case the disease to be prevented is cancer and tumor, respectively, the medicament and pharmaceutical composition according to the present invention is administered to a patient or a person who shall be prevented from developing the disease. Using the double-stranded nucleic acid according to the present invention, any cell which has a tendency to show any of the scenarios described herein, such as, e.g. loss of gene or loss of function of, e.g. tumor suppressor, or has already undergone such loss, can be addressed and thus, finally, inhibited or lysed. Due to this, the organism is freed from such cell which could be the origin of a tumor or cancer disease, particularly in case of monoclonal tumors and cancers. In connection therewith it is to be noted that the cell is usually a diploid cell. Although any genetic information is thus basically present in double, the genetic information may vary between the two sets of genetic information as will the transcript of the respective gene. However, the presence of a transcript of a functionally inactive protein involved in the particular disease contemplated, preferably a tumor suppressor in case of tumor and cancer, respectively, can be addressed according to the present invention and thus the stress response triggered which ultimately results directly or indirectly in the growth inhibition, apopotises or lysis of the respective pathological cell.

As used herein and if not indicated to the contrary herein, the double-stranded nucleic acid according to the present invention has the following structure: The nucleic acid comprises a double-stranded structure and the double-stranded structure comprises a first strand and a second strand, wherein the first strand comprises a first stretch of contiguous nucleotides and the second strand comprises a second stretch of contiguous nucleotides. This design is also referred to as basic design. Preferably, the double-stranded nucleic acid is a double-stranded ribonucleic acid. However, it is also within the scope of the present invention, that the double-stranded nucleic acid is a double-stranded deoxyribonucleic acid. In a further embodiment, the double-stranded nucleic acid comprises a first strand, with the first stretch and first strand, respectively, being a ribonucleic acid and the second strand and the second stretch, respectively, being a deoxyribonucleic acid. In an alternative embodiment, the first stretch and the first strand, respectively is a deoxyribonucleic acid and the second stretch and second strand, respectively, is a ribonucleic acid.

According to the present invention, the basic design of the double-stranded nucleic acid according to the present invention can in principle be modified as follows, particularly for the second scenario which is disclosed herein, i.e. where the target nucleic acid is present in the transcription system, but the stress response as defined herein is triggered by the lack of complementarity if the antisense strand as preferably provided by the first strand and first stretch, respectively, of the double-stranded nucleic acid according to the present invention, is not fully complementary to the target nucleic acid due to one or several mismatches. Any mismatch is formed by a pair of nucleotides on opposite strand which are not properly base paring. In the first modification the first stretch of contiguous nucleotides of the double-stranded nucleic acid according to the present invention is complementary to the target nucleic acid except one or several nucleotides creating such mismatches. In other words, this kind of first stretch is not complementary to the target nucleic acid, more preferably to any of the nucleic acids of the transcription system such as the transcriptome of a cell. In any case, it is preferred that the design of the thus modified double-stranded ribonucleic acid according to the present invention is RNA interference response-negative. The second stretch of contiguous nucleotides of the double-stranded nucleic acid according to the present invention is in one embodiment then identical to the target nucleic acid except one or several nucleotides at the positions corresponding to the positions of the mismatches. The mismatch(es) result(s) in a bulge or loop structure if the first stretch of contiguous nucleotides is base paired with a target nucleic acid, more preferably any nucleic acid of the transcription system. In a preferred embodiment, the number of mismatches is 1 to 10, preferably 2 to 8 and more preferably 2 to 4, most preferably 4, wherein such mismatches are preferably arranged in the first stretch and second stretch, respectively. As used herein, a range specified from a first figure to a second figure means that any of the figures comprised by that range. If, for example, a range is from 1 to 4 this means that what is actually disclosed is any integer from 1 to 4, i. e. 1, 2, 3 and 4.

In a preferred embodiment of the present invention the double-stranded nucleic acid according to the present invention having the basic design or a modification thereof, comprises on the first strand a sequence or stretch of nucleotides which is 14 or less nucleotides in length and said sequence or stretch is perfectly matching with the target nucleic acid sequence as defined herein. In other words, the double-stranded nucleic acid according to the present invention comprises on the first strand, which is preferably the antisense strand, a sequence which is maximum 14 nucleotides in length and such sequence is perfectly matching to a target nucleic acid. The additional or other nucleotides comprised by the first strand or the first stretch of the double-stranded nucleic acid according to the present invention are not perfectly base paring thus giving rise to mismatches and such mismatches provide for the characteristic of the double-stranded nucleic acid according to the present invention that the overall first stretch is not complementary to a target nucleic acid as defined herein. This characteristic can be realized under both scenarios as disclosed herein, namely that the target gene is not present at all in the cell and more precisely in the transcriptome of the cell which is to be treated using the double-stranded nucleic acid according to the present invention, and that the target gene is present, however, the double-stranded nucleic acid according to the present invention triggers the stress response, wherein such stress response and the triggering thereof, respectively, is preferably caused by the first stretch of the double-stranded nucleic acid according to the present invention not being complementary to the target nucleic acid.

The same considerations as above are applicable to the second strand of the double-stranded nucleic acid according to the present invention, wherein the criterion is identity rather than complementarity.

In an alternative embodiment the first stretch of contiguous nucleotides is not fully complementary to the target nucleic acid, whereas the second stretch is fully identical to the target nucleic acid. Such a construct of the double-stranded nucleic acid according to the present invention would, however, result in a mismatched or bulged double-stranded structure similar to the one observed between the antisense strand and the target nucleic acid. It is, however, also within the present invention that the first stretch and the second stretch are completely, i.e. perfectly, base pairing which would mean that under the assumption that the first stretch is not completely base pairing with the target nucleic acid because one or several nucleotides are not complementary to the target nucleic acid, the second stretch is thus not fully identical with the target nucleic acid. In a preferred embodiment, the position of the mismatch of the first stretch with regard to the target nucleic acid is located on the corresponding position of the second stretch not being completely identical to the target nucleic acid. It is to be acknowledged that the afore described embodiments with regard to their mismatches can also be used in case the target nucleic acid is not present at all, such as in case the respective mRNA member of the transcriptome is not present, preferably under the proviso that still this kind of molecule is suitable to trigger the stress response outlined above.

The mismatches between the first stretch and the target nucleic acid can be arranged such as to truncate the first stretch of nucleotides thus as to reduce the length of the complementary stretch, or the mismatches can be located within the first stretch which means preferably at a position different from the 5′ and the 3′ end. The same applies in principle also to the number and localisation of the “mismatches” at the level of the second stretch of contiguous nucleotides which are, in general, characterised as being identical to the target nucleic acid.

It is within the present invention that the number and localisation of the mismatches can be varied, wherein such variation is deemed to be within the scope of the present invention as long as the above described effect is observed, namely that the factors mediating RNA interference, preferably an expression system, provide for a signal which results in the stress response.

About the length of the double-stranded nucleic acid according to the present invention, it is as little as 15 and as much as several hundred oligonucleotide pairs. In an embodiment the first strand and the second of the double-stranded nucleic acid according to the present invention are of the same length. However, in an alternative embodiment the first strand and the second strand of the double-stranded nucleic acid according ot the present invention are different in length. In an embodiment, the first stretch is of the same length as the first strands and the second stretch is of the same length as the second strand. It is acknowledged that the minimum length of the first stretch and/or of the second stretch is similar to those lengths realized in the field of RNAi as known in the art and as also specified herein. In an alternative embodiment, the length of the first stretch and/or second stretch is 19 to 40 nucleotides, preferably 19 to 30 and more preferably 21 to 30 and most preferably 21 to 27 oligonucleotides, wherein this applies also to the first strand and the second strand, respectively. It is acknowledged that preferably the length of the first stretch and/or the second stretch is less than the length of an oligonucleotide triggering the PKR reaction which is responsible for the non-specific effects on gene expression resulting in an interferon response. On the other hand, the first stretch and/or the second stretch and the first strand and/or the second strand, respectively, can be as long as several hundred oligonucleotides, wherein under these circumstances preferably only a part of these stretches and strands will act in the sense described herein for the double-stranded oligonucleotide according to the present invention, i.e. triggering the stress response. The longer the first and/or second stretch is, the more likely it is that they as a whole or part thereof find a target sequence in the transcriptome and thus triggering specific RNA interference which is preferably to be avoided when applying the double-stranded nucleic acids according to the present invention. It is to be understood that what has been said in connection with the first and second stretch, respectively, applies also to the first strand and second strand, respectively. For reasons of clarity it shall be mentioned that in some embodiments of the present invention, this kind of interferon response can be tolerated. In a further aspect the present invention is also related to the use of the double-stranded nucleic acid for the manufacture of a medicament, wherein such medicament is for the stimulation of the immune system of a patient, wherein preferably the patient is in need thereof. The double-stranded nucleic acid according to the present invention when designed in accordance with the other aspects of the present invention, more particularly when used for the treatment of diseases such as tumors, is deemed to be suitable to elicit an unspecific immune response. Such unspecific immune response can be advantageous and thus be used as a supporting therapy for disease where such unspecific immune response is suitable to increase the efficacy of the main therapy. Such main therapy can preferably be any tumor therapy or any therapy involving vaccination such as, e.g. vaccination for the treatment of infectious diseases or treatment of cancer and tumor diseases.

Nevertheless it is preferred that the length of the double-stranded nucleic acid according to the present invention does not induce an interferon response. More preferably, the length thereof and more particularly the length of the first and/or the second stretch is 15 to 30, more preferably 17 to 25, even more preferably 19 to 23 and most preferably 21 to 23 nucleotides, when the design principles as outlined above for the double-stranded nucleic acid according to the present invention.

Preferably, the double-stranded nucleic acid according to the present invention comprises at the 3′-end of the first and second strand one or several deoxyribonucleotides, preferably two deoxyribonucleotides and most preferably 2 TT. Alternatively, this kind of modification can be present at the 5′-end of the first and the second strand of the double-stranded nucleic acid according to the present invention. In a further preferred embodiment, this kind of modification, preferably consisting of one or several, more preferably two deoxyribonucleotides, is attached at the 3′-end of the first strand which is preferably the antisense strand, and one or several, more preferably two deoxyribonucleotide(s) are attached at the 5′-end of the second strand, i. e. which is preferably the sense strand, of the double-stranded nucleic acid according to the present invention. In a further alternate embodiment of the double-stranded nucleic acid according to the present invention, the two ends of the first and of the second strand are blund ended. The length of the double-stranded nucleic acid can be any of the lengths described herein insofar. However, the length of the first stretch and second stretch, respectively is preferably from 19 to 30 nucleotides, more preferably from 21 to 27 nucleotides, and even more preferred from 21 to 23 nucleotides. In a still preferred embodiment, the number of mismatches, if any, is from 1 to 4 with 2 to 4 and 3 or 4 mismatches being more preferred.

The further design of the double-stranded nucleic acid according to the present invention can be as follows. Preferably, the nucleotides of the double-stranded nucleic acid according to the present invention are ribonucleotides. Alternatively, In principle, any of the nucleotides contained in the double-stranded nucleic acid can comprise a modification wherein said modification is preferably selected from the group comprising nucleotides being an inverted abasic and nucleotides having an NH₂-modification at the 2′-position. Any further modification of the individual nucleotide being contained in the double-stranded nucleic acid according to the present invention, can be selected from the group comprising amino, fluoro, methoxy, alkoxy and alkyl. Preferably, alkoxy is ethoxy. Also preferably alkyl means methyl, ethyl, propoyl, isopropyl, butyl and isobutyl, wherein such modification is located at the 2′ position of the sugar moiety of the nucleotide. Regardless of the type of modification, the nucleotide is preferably a ribonucleotide.

In addition or alternatively to the afore-mentioned modifications, each and any of the nucleotides can be modified at the phosphate moiety of the nucleotide. Such modification can be the presence of a phosphothioate. It is within the present invention that the individual nucleotides of the stretches and strands, respectively, of the double stranded nucleic acid according to the present invention are linked through a phosphodiester linkage or through a phosphothioate linkage, or a combination of both along the length of the nucleotide sequence of the individual strand and stretch, respectively.

A further from of modification which the strands, either each single strand or both strand of the double-stranded nucleic acid according to the present invention is any modification of the terminal nucleotides, i.e. the most 3′ or the most 5′ nucleotide. Such kind of modification can be selected from the group comprising inverted (deoxy) abasics, amino, fluoro, chloro, bromo, CN, CF, methoxy, imidazole, caboxylate, thioate, C₁ to C₁₀ lower alkyl, substituted lower alkyl, alkaryl or aralkyl, OCF₃, OCN, O—, S—, or N-alkyl; O—, S—, or N-alkenyl; SOCH₃; SO₂CH₃; ONO₂; NO₂, N₃; heterozycloalkyl; heterozycloalkaryl; aminoalkylamino; polyalkylamino or substituted silyl, as, among others, described in European patents EP 0 586 520 B1 or EP 0 618 925 B1. As used herein and more particularly in connection with the aforementioned modification, alkyl or any term comprising “alkyl” means any carbon atom chain comprising 1 to 12, preferably 1 to 6 and more preferably 1 to 2 C-atoms.

A further end modification is a biotin group. Such biotin group may preferably be attached to either the most 5′ or the most 3′ nucleotide of the first and/or second strand or to both ends. In a more preferred embodiment the biotin group is coupled to a polypeptide or a protein. It is also within the scope of the present invention that the polypeptide or protein is attached through any of the other aforementioned end modifications. The polypeptide or protein may confer further characteristics to the inventive nucleic acid molecules. Among others the polypeptide or protein may act as a ligand to another molecule. If said other molecule is a receptor the receptor's function and activity may be activated by the binding ligand. The receptor may show an internalization activity which allows an effective transfection of the ligand bound inventive nucleic acid molecules. An example for the ligand to be coupled to the inventive nucleic acid molecule is VEGF and the corresponding receptor is the VEGF receptor.

The aforementioned modifications, preferably those related to the 2′ position of the nucleotides, more preferable of the ribonucleotide, can be applied to the double-stranded nucleic acid according to the present invention in a certain pattern. One such pattern is a spatial pattern as described in international patent application PCT/EP 03/08666. More particularly such spatial pattern is such that the double-stranded ribonucleic acid comprises a double stranded structure, wherein the double-stranded structure comprises a first strand and a second strand, wherein the first strand comprises a first stretch of contiguous nucleotides and wherein said first stretch is at least partially complementary to a target nucleic acid, and the second strand comprises a second stretch of contiguous nucleotides and wherein said second stretch is at least partially identical to a target nucleic acid, which is characterised in that said first strand and/or said second strand comprises a plurality of groups of modified nucleotides having a modification at the 2′-position wherein within the strand each group of modified nucleotides is flanked on one or both sides by a flanking group of nucleotides wherein the flanking nucleotides forming the flanking group of nucleotides is either an unmodified nucleotide or a nucleotide having a modification different from the modification of the modified nucleotides. In a preferred embodiment, the group of modified nucleotides comprises one nucleotide and the group of unmodified nucleotides comprises one nucleotide. Furthermore, it is preferred that the non-modified nucleotide is a ribonucleotide and that the modified nucleotide is a ribonucleotide modified as disclosed herein, with the modification being a methoxy group at position 2′ of the ribose moiety of the nucleotide. In a further preferred embodiment, the 5′ end of the first strand, i.e. the antisense strand starts with a modified nucleotide, whereas at the corresponding position of the sense strand the nucleotide at the 3′ end is a non-modified nucleotide.

A further from of modification can be based on discriminating whether the individual nucleotide is a purine or a pyrimidine. In one alternative, any purine nucleotide of the double-stranded nucleic acid according to the present invention is modified as described herein, and in another alternative, any pyrimidine nucleotide of the double-stranded nucleic acid according to the present invention is modified as described herein. Modification patterns of this type are, among others described in international patent application PCT/US 03/70918. More particularly, the pyrimidine nucleotides of the first strand and stretch, respectively, i.e. the antisense strand of the double-stranded nucleic acid according to the present invention are 2′deoxy-2′-fluoro pyrimidine nucleotides and the purine nucleotides of the first strand and stretch respectively, i.e. the antisense strand of the double-stranded nucleic acid according to the present invention are 2′-O-methyl purine nucleotides.

It is also within the present invention that the two strand forming the double-stranded nucleic acid are linked to each other. Such linkage can consist of a single linkage or of a plurality of linkages. Preferably, the linkage occurs between the 5′ end of one of the two strands and the 3′ end of the other strand forming the double-stranded nucleic acid according to the present invention. This kind of linkage is preferably made through a linker. Such linker preferably consists of a multitude of nucleotides. Alternatively, such linker consists of a non-nucleotide polymer such as a peptide, LNA, PNA or PEG.

It is within the present invention that the double-stranded nucleic acid according to the present invention is chemically synthesized and subsequently formulated. Such formulation can be any of the formulations known in the art. A preferred formulation comprises at least one lipid, preferably one lipid and a helper lipid. Even more preferably the lipid is a cationic lipid such as the lipid used in the examples, and the helper lipid is a helper lipid as described in the examples. The formulation preferably exhibits the features as the formulation described in the examples.

It is also within the present invention that the double-stranded nucleic acid according to the present invention is synthesized in vitro or in vivo. For such purpose a genetic construct is provided the transcription of which results in the two strands forming the double-stranded nucleic acid according to the present invention. The genetic construct preferably comprises a promoter under the control of which a sequence coding for the strand(s) is transcribed. The genetic construct can be such that each individual strand is transcribed from its own promoter, wherein the promoters are identical. In an alternative embodiment, the promoter is the same. In a further embodiment, the same promoter controls the transcription of both strands and the sequences coding therefor. In a still further embodiment the genetic transcript comprises the sequences coding for the two strands of the double-stranded nucleic acid according to the present invention, wherein both sequences are linked to each other trough a sequence which allows the formation of a loop or hairpin after the transcription of the genetic transcript or a part thereof. Under such condition the transcript can fold back such that the two complementary strands, i.e. the first and the second strand of the double-stranded nucleic acid according to the present invention can base pair and are linked through the loop sequence. This kind of technology is, among others described in international patent application PCT/AU 99/00195 or international patent application PCT/IB 99/00606, there disclosure of which is incorporated herein by reference. It is to be acknowledged that the respective promotors are known in the art and, among others described in Current Protocols in Molecular Biology; Ausubel F. M., Brent R, Kingston R. E., Moore D. D., Seidman J. G., Smith J. A. and Struhl K. (1996); J. Wiley and Sons, New York.

In a further embodiment, the double-stranded nucleic acid according to the present invention can also be designed such as any RNAi molecule or siRNA molecule as described and known in the prior art, provided that the absence of the target nucleic acid in the transcription system to which the double-stranded nucleic acid according to the present invention is to be administered or in which double-stranded nucleic acid according to the present invention is to be expressed or active, as defined herein is given. A further form of siRNA molecules which may be used according to the technical teaching of the present invention is siNA as described in international patent application PCT/US 03/05346.

In connection with the afore-mentioned possible modifications it is preferred that such modification still allows a double-stranded molecule according to the present invention and preferably in case such molecule is designed as any siRNA molecule or any RNAi molecule, to be active in the sense that an RNA interference response would be caused, if such modification was realized on an RNAi molecule or a siRNA molecule which molecules have and find a target nucleic acid in a cell. In other words, the modification as possibly realized on the double-stranded nucleic acid according to the present invention is preferably any modification which, if applied to RNAi molecules and siRNA molecules according to the prior art, is still able to cause an RNA interference response. Without wishing to be bound by any theory, the present inventors assume that the stress response as described herein and used for, e.g. apoptosis and cell lysis, respectively, mediated through the double-stranded nucleic acid according to the present invention might still, at least to a certain extent, interact with component(s) of the RNA interference machinery such as the RISC complex, and the requirements of these components on the acceptability of a modification are also applicable to the double-stranded nucleic acid according to the present invention. In so far the afore-mentioned provides already for a suitable assay which allows which kind of modification(s) are preferably also acceptable to the double-stranded nucleic acid according to the present invention.

In the first scenario of the present invention as outlined above, the double-stranded nucleic acid according to the present invention is directed to a target nucleic acid, which as such is not present in a transcription system such as a cell. Preferably, the nucleic acid is not present as an mRNA, a hnRNA or other transcription product. More particularly, the double-stranded nucleic acid according to the present invention is not directed to any of the respective nucleic acids contained in said expression system, wherein the entirety of this nucleic acid is also referred to as the transcriptome or as the target nucleic acid herein in general. Under these circumstances, the double-stranded nucleic acid according to the present invention being directed to a target nucleic acid means that the first stretch of the first strand is complementary thereto to the extent defined herein and the second stretch of the second strand is identical to the extent defined herein. Due to the mechanism described above, the interference machinery will not find a target nucleic acid and thus trigger the stress response. In order for the cell not to find a target nucleic acid, it is essential that the double-stranded nucleic acid according to the present invention is designed such that no other nucleic acid of the transcriptome could actually act as a target thus triggering an RNA interference response. This can be achieved by bioinformatics. Typically, the possible target sequences are compared to the overall transcriptome and the respective stretch be designed in terms of nucleotide sequence such that it does not comprise a sequence matching with another nucleic acid thus not leading to an off-target effect which would thus not allow to see the result of a negative RNA interference response or, in therapeutic application go along with, possibly, undesired side effects. Particular in case of tumor diseases, the target nucleic acid is a tumor suppressor which, preferably, is not part of the transcriptome of the respective cell line. Accordingly, preferred tumors to be treated by this method and for which the double-stranded nucleic acid according to the present invention can be used, are tumors which are tumor suppressor-negative. Preferred tumor suppressors are PTEN, p53, p21 and Rb, although the present invention is not limited thereto. It is also within the present invention that not only one type of double-stranded nucleic acid is used for the purposes disclosed herein such as manufacture of a medicament, but two or even more than two of such double-stranded nucleic acids, wherein at least two of the double-stranded nucleic acids are addressing two different tumor suppressors which results in an increased efficacy of the stress response itself or of the triggering of the stress response.

It is within the present invention that the pathological cell can be tumor suppressor-defective. This means that the pathological cell does not produce or have a tumor suppressor. Due to this lack of tumor suppressor, cancer or tumor may arise. The tumor suppressor-defective state, which is also a loss of function state, can be generated in different ways. First, the cell can be devoid of any genetic information for said tumor suppressor. Such lack will result in the cell not to have any tumor suppressor at all and, accordingly, no transcript of such gene. Second, the cell can be devoid of any functional tumor suppressor. A functional tumor suppressor as preferably understood herein, is any tumor suppressor which is active in suppressing a tumor. The lack of any functional tumor suppressor can result from the gene and transcript thereof, respectively, having one or several mutations which result infunctionally inactive tumor suppressor. Such mutation can, e.g., be a point mutation or a deletion mutation. The transcript having these mutation(s) is regarded as target nucleic acid as defined herein and thus allows a respective design of a double-stranded nucleic acid according to the present invention.

It is to be acknowledged that when using this kind of double-stranded nucleic acid with one strand being complementary to a tumor suppressor, in a non-pathological cell, i. e. in a cell which is not involved in the disease, such as tumor, of the organism to be treated with the kind of double-stranded nucleic acid according to the present invention, this will result in a knock-down of the tumor suppressor. Such knock-down is most likely mediated through an RNA interference response. However, although this may be regarded as some sort of side effect, it is deemed not critical for the treatment regimen according to the present invention given the fact that the double-stranded nucleic acids according to the present invention are active only in a transitory manner which means that once the toxic side effects resulting, among others, in apoptosis, are triggered, the respective pathological cells are eliminated whereas the non-pathological cells are no longer exposed to this kind of agent and thus the knock-down of the tumor suppressor will be stopped. In other words, the tumor suppressor is only knocked down for a limited period of time in those cells which are not tumor suppressor negative as the respective tumor cells, which will not allow the cell to develop a pathological condition.

The same applies also to the strategy underlying the second scenario disclosed herein related to the use of the double-stranded nucleic acid according to the present invention having one or more mismatches relative to the target nucleic acid wherein a respective target nucleic acid is present in a cell, more particularly in a pathological cell or a cell involved in any pathological or diseased condition. Again, the double-stranded nucleic acid according to the present invention is preferably designed such as not to create an RNA interference response, but to trigger the stress response as described herein, whereas those cells not involved in the diseased condition or having a disposition to develop such condition will experience a temporary knock-down only. Of course, any other off-target effects shall be avoided which can be realised by selecting the appropriate conditions and sequences, respectively using bioinformatics.

In a further aspect the present invention is related to a method for the manufacture and/or design of a medicament for the diseases described herein, more particularly tumors, tumor diseases, cancer and cancer diseases as well as any disease which is characterised by a loss of function or by a loss of gene or a disease which involves at least one SNP as defined herein. According to the method the disease to be treated, more particularly the cells involved in said disease, are characterised in terms whether they express the factor, preferably the peptide and protein, respectively, responsible for such disease. If said cells do not express such factor and/or do not comprise a transcript for or encoding such factor, a double-stranded nucleic acid the present invention is designed such as to avoid any off-target effect, wherein the transcriptome of the cell does not comprise the mRNA or hnRNA of the respective factor. Alternatively, if the respective factor is a mutated form of the factor found in normal, i. e. healthy cells, the double-stranded RNA is designed such as to be complementary to the mRNA or hnRNA or other transcript of the factor in the mutated form, wherein as many mismatches are introduced such as to allow the generation of the stress response as described herein, whereas preferably no positive RNA interference response is generated and, again, any off-target effects are avoided by designing the sequence accordingly. This kind of sequence design can be performed by using bioinformatics as known to the ones skilled in the art. It is to be acknowledged that any off-target effect is to be avoided, however, it is in principle sufficient if the off-target effects are reduced, or occur in connection with other components or factors of the cell which are deemed of not doing any harm to the cell. Preferably, however, there is no off-target effect caused by the respective sequence.

The invention is now further illustrated by reference to the figures and examples from which further features, embodiments and advantages of the present invention may be taken.

### EXAMPLE 1

### Reduction of PTEN Protein in HeLa Cells with Different Knock-Down Technologies

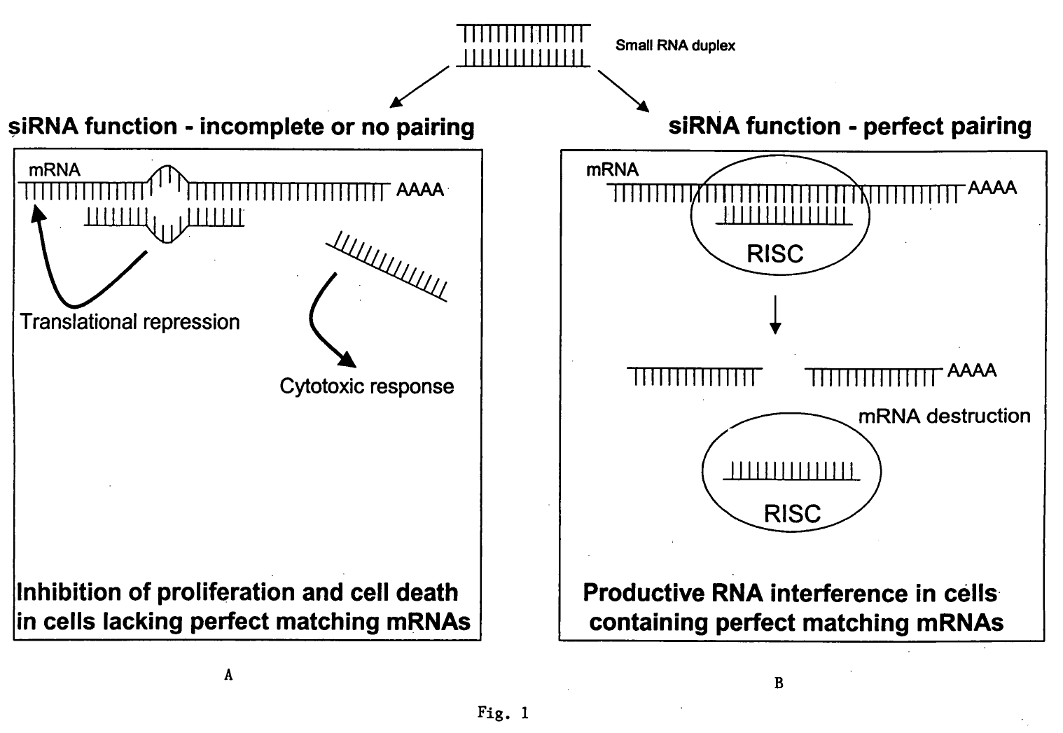

In order to asses the reduction of PTEN protein in HeLa cells with different knock-down technologies, siRNA constructs and a gene block, i. e. an antisense construct was prepared. The respective sequences and design principles can be taken from FIG. 1A. It is to be noted that the siRNA constructs against PTEN are designated as PTEN1, PTEN2 and PTEN3, respectively. Also, siRNA constructs were created having in each case four mismatches which are referred to as PTEN1 MM, PTEN2 MM and PTEN3 MM. It is to be acknowledged that the arrangement of the mismatches in these siRNA constructs can be used for any other siRNA, i. e. that this arrangement is not limited to the use for PTEN but may be used for other target nucleic acid sequences as well. The mismatches compared to the PTEN mRNA sequence, i. e. the target sequence, are marked by arrows. Additionally, the antisense construct was created which has at its 5′ and 3′ ends an inverted abasic and comprises in the core a stretch of nine desoxyribonucleotides flanked by six oligonucleotides at either end. The respective antisense construct is referred to as PTEN1 GB. Also for this kind of antisense molecule a mismatching antisense version was designed which comprises a total of four mismatches compared to the target PTEN mRNA.

HeLa cells were maintained in minimum essential medium Eagle (EMEM with 2 mM L-glutamine, Earle's BSS 1 mM sodium pyruvate, 0.1 mM non-essential amino acids, 10% fetal calf serum (FCS). Synthetic siRNA and antisense transfections, i. e. GeneBlocs transfections were carried out in 10-cm plates (at 30% to 50% confluency) by using L8 lipid (Atugen, Berlin). HeLa cells were transfected by adding pre-formed 5× concentrated complex of siRNAs and lipid in serum-free medium to cells in complete medium. The total transfection volume was 10 ml for cells in 10 cm plates. The final lipid concentration was 0.8 to 1.2 μg/ml depending on cell density. For immunoblots, cells were lysed and aliquots of the cell extracts containing equal amounts of protein were blotted on Nitrocellulose membrane and analyzed by standard methods using murine monoclonal anti-PTEN antibody (Santa Cruz Biotechnology) and the murine monoclonal anti-p110alpha antibody for assessing equal loading (Klippel, 1994).

The effects of the various constructs were tested on HeLa cells and the results depicted as immunoblots.

In principle, whenever PTEN is knocked down, the level of the phosphorlyated form of Akt should increase. However, it is also known that phospho-Akt shows an increased expression under stress conditions. Comparing the various siRNA constructs it can be taken from FIG. 1B that a knock-down is generally created, although the downstream read-out, namely phospho-Akt is no longer consistent with the underlying biological signalling cascade. This effect is solely shown by the siRNA constructs, whereas the antisense construct does not show this kind of unpaired reaction at the expression level of HeLa cells. In other words, siRNA mediated PTEN knock-down does not necessarily result in stimulation of Akt phosphorylation. This result suggests that the presence of the siRNA molecules in the cell may prevent a normal signal transduction. This leads to the conclusion that, as illustrated in connection with PTEN3 MM, double-stranded ribonucleic acid designed according to the present invention can result in a stress reaction including cytotoxic response and apoptosis and impaired cell signalling.

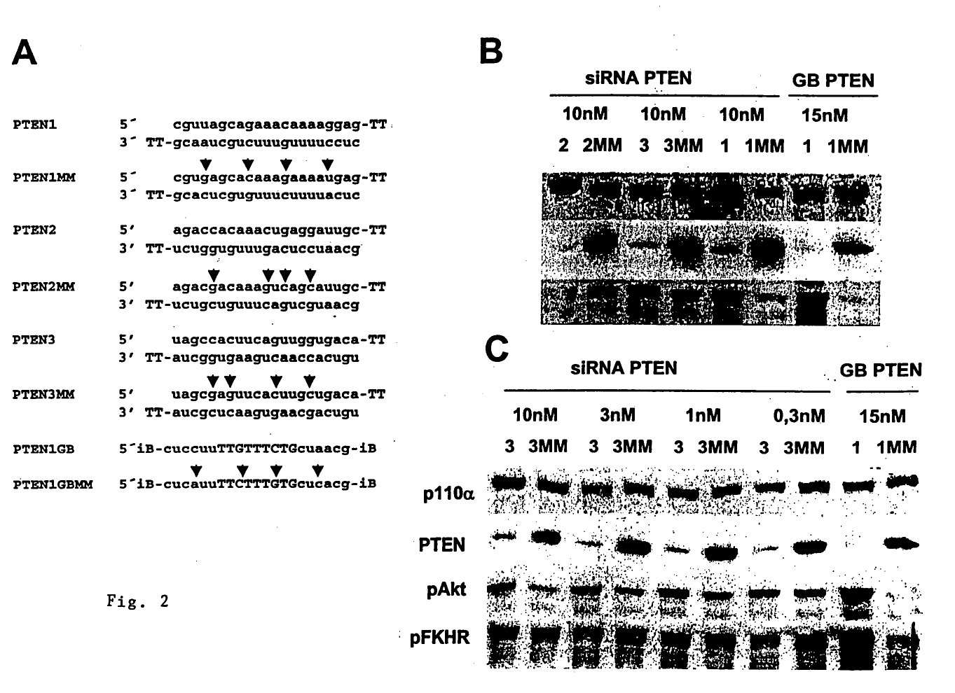

Additionally, as an even further downstream possible read-out pFKHR was used. pFKHR is a further transcription factor. In this experiment the PTEN3 construct which turned out to be particularly interesting, was subject to a titration experiment confirming the results depicted in FIG. 2B. This titration experiment demonstrates that the impaired cell signalling is not caused by excessive intracellular siRNA as proven by the fact that it can not be reduced by delivery of limited amounts of siRNA.

### EXAMPLE 2

### Testing the Impact of Various Knock-Down Technologies in an Affymetrix Experiment

An Affymetrix experiment was designed using the nucleotides described in connection with example 1, wherein GB1 corresponds to PTEN1 GB, GB1 MM corresponds to PTEN1 GB MM, siRNA1 corresponds to PTEN1 and siRNA 1 MM corresponds to PTEN1 MM.

The selection criteria for the called probe sets were that there was a presence in both treated and untreated cells and a change in the intensity of the signal of at least 2.5 fold.

For microarray experiments, RNA from cells treated with corresponding GB/siRNA was prepared and biotinylated cRNA probes were generated and hybridized to Affymetrix Human Genome U95 set (Chip HGU133A and B) according to the manufacturer's protocol (Affymetrix, Santa Clara, Calif.-USA). Absolute analysis of each chip and comparative analysis of samples were carried out by using the Affymetrix software (Microarray suite, Version 5.0).

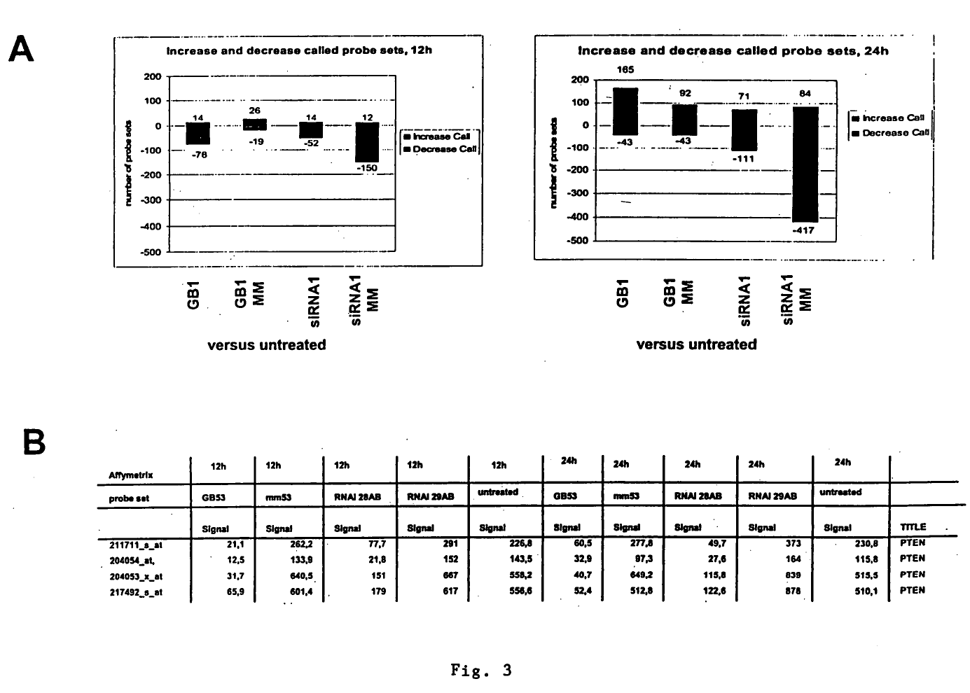

The results of these experiments are depicted in FIG. 3A. From the Affymetrix experiment, about 6,000 probe sets were analysed upon treatment of the respective HeLa cells with the aforementioned knock-down constructs. In any case it can be taken from the result, that there is a significant decrease in called probe sets after twelve hours, irrespective of the particular sequence. It is to be noted that the siRNA construct has a significant decrease of called probe sets after twelve hours. In any case, the antisense mediated effects are less pronounced than the one of the siRNA constructs.

After 24 hours of incubation the respective HeLa cells shows in case of antisense constructs, irrespective whether with our without mismatches, a significant increase of called probe sets which is thus a reversion of the situation observed after twelve hours. This is in contrast to the situation with siRNA constructs which still show a significant decrease of called probe sets. In connection therewith even the mismatch siRNA corresponding to the double-stranded ribonucleic acid constructs according to the present invention, show a significant decrease in called probe sets.

### EXAMPLE 3

### Inhibiton of Tumor Cell Growth by dsRNA Not Pairing to the Transcriptome of the Tumor Cells

In the present example an experiment is described where the inhibition of tumor cell growth is caused by dsRNA not pairing to the transcriptome of the tumor cells.

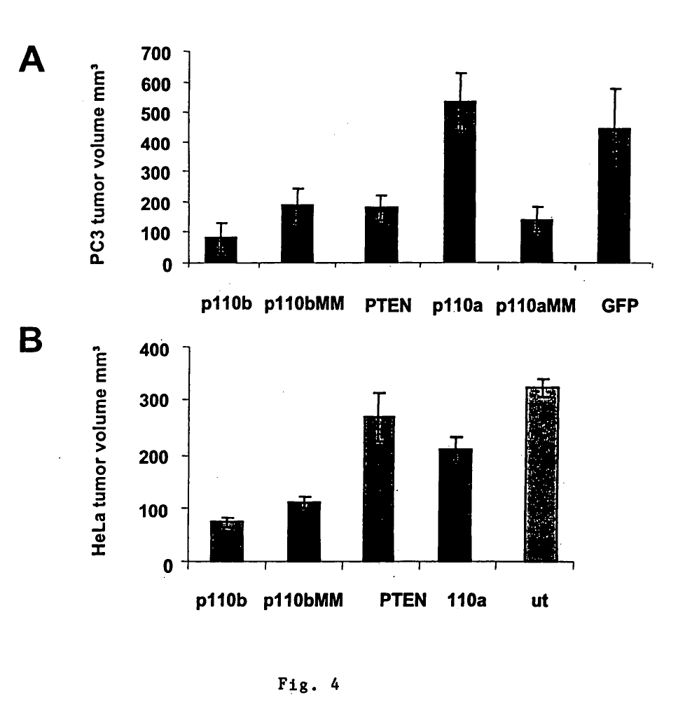

The basic procedure of this experiment can be summarised as follows. Human prostate carzinoma cells (PC3) or HeLa cells expressing short hairpin RNA (shRNAs) were generated. A total of 5×10⁶ cells were injected in 8 nu/nu mice (subcutan HeLa, intraprostatic PC3). The mice were sacrified 56 days post injection for PC3 and 10 days post injection for HeLa. The end point was growth of implantation tumor The results thereof are depicted in FIG. 4.

FIG. 4A shows the tumor volume of PTEN (−/−) prostate carzinoma cells (PC3) stably transfected with the indicated shRNA expression constructs. PC3 cells stably transfected with GFP expression plasmid were included as control of the orthotopic tumor model. The respective sequences are:

| | |
|---|---|
| PTEN | guucacuguaaagcuggaaaggg aaaaaaaaaaaa |
| | cccuuuccagcuuuacaguga |
| PTEN MM | guucacucuaaaggugcaaacgg aaaaaaaaaaaa |
| | ccguuugcaccuuuagaguga |

In this particular experiment siRNA against the two subunits of P13K, namely p110b and p110a were designed. Given the importance of the p110b subunit, also sometimes referred to as p110beta, a siRNA construct against this target should be particularly efficient in terms of reducing the tumor volume as also confirmed by this experiment. However, it is surprising to see that also the siRNA which is a p110a MM is nearly as effective as the tumor specific p 110b siRNA, whereas the p110a is the negative control for p110b. Additionally, the siRNA construct which is a mismatched form of the p110b still shows significant inhibition of the PC3 tumor cell volume. This effect confirms the understanding that even if the double-stranded ribonucleic acid according to the present invention is designed so as not to interact with an mRNA or another element of the transcriptome of the PC3 tumor cells, this leads to a cytotoxic response and apoptosis, respectively, as may be taken from the reduction in tumor volume.

The surprising finding underlying the present invention that also siRNA constructs are, in principle, effective in inducing a cytotoxic response and apoptosis in case the siRNA is directed against a member of the transcriptome which is not present in the respective cell line and does not trigger an RNA interference response with other nucleic acids, is confirmed by the effect of the PTEN construct which is about as effective as the highly specific p110b siRNA construct.

A further result from this experiment is depicted in FIG. 4B which shows the tumor volume of PTEN (+/+) HeLa cells stably transfected with the indicated shRNA expression plasmid used in the subcutane tumor model. Here, the PTEN siRNA constructs find a target because HeLa cells are PTEN positive with regard to both alleles thus not inducing that cytotoxic response. Nevertheless, the p110b MM construct, i. e. the siRNA construct which is specific for p110, however, has several mismatches in compliance with the design principles disclosed herein, which do not allow for a proper, i. e. positive RNA interference response, also results in a significant reduction of the HeLa tumor volume.

### EXAMPLE 4

### Reduced PC-3 Cell Proliferation Induced by Transfection of dsRNA Molecules

This example was performed in order to evaluate the impact of the modification pattern of a dsRNA molecule as well as the impact of the presence and absence, respectively, of the target nucleic acid of the dsRNA introduced.

The following dsRNA molecules were induced:

| | |
|---|---|
| PTENA | 5′- cuccuuuuguuucugcuaacg-TT |
| PTENB | 3′- TT-gaggaaaacaaagacgauugc- |
| PTENAMM | 5′- cucauuuucuuugugcucacg-TT |
| PTENBMM | 3′- TT-gaguaaaagaaacacgagugc |
| PK 71A | 5′- cuucucgcaguacaggcucuc-TT |
| PK 71B | 3′- TT-gaagagcgucauguccgagag |
| NM_013355 | |
| PTENAV15 | 5′- cuccuuuuguuucugcuaacg |
| PTENBV15 | 3′- gaggaaaacaaagacgauugc |
| PTENAV1 | 5′- cuccuuuuguuucugcuaacg |
| PTENBV1 | 3′- gaggaaaacaaagacgauugc |
| PTENAV10 | 5′- uaaguucuagcuguggugg |
| PTENBV10 | 3′- auucaagaucgacaccacc |

Bold and underlined=2′-O-methyl modification

Capital=Desoxy nt

The efficacy of the various dsRNA molecules was assessed in a proliferation assay on 10 cm plates using PC-3 cells.

Proliferation Assay on Plastic Dishes

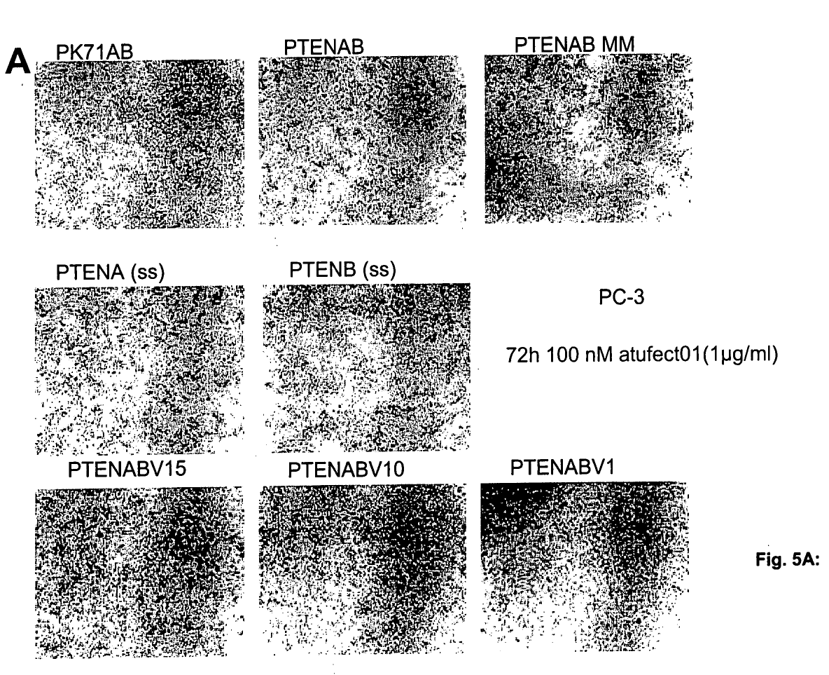

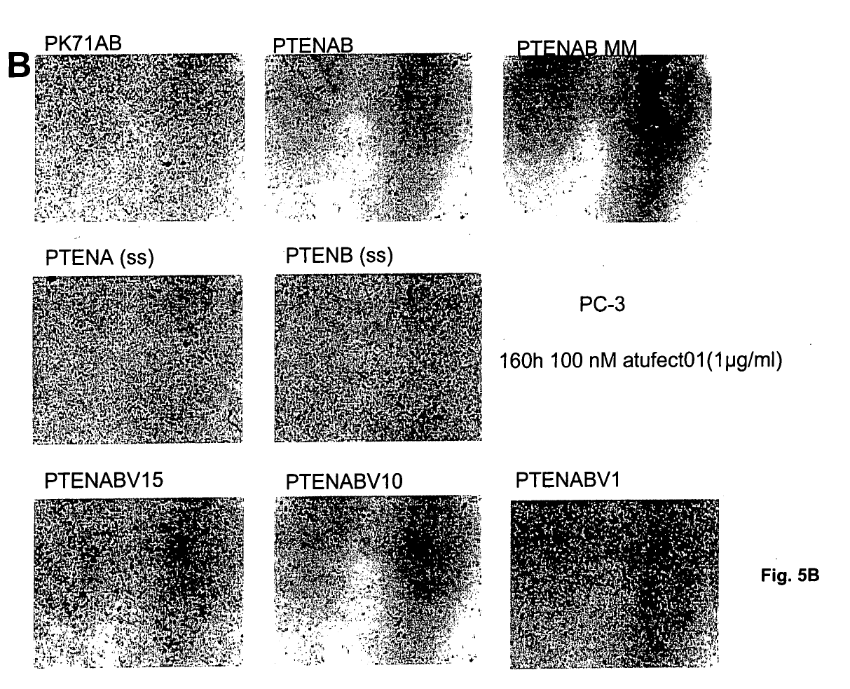

30-50% confluent PC-3 cells were transfected with the respective dsRNA molecule (see Example 1). After incubation photographs were taken at 72 h which are FIG. 5A (72 h incubation) and at 160 h which are FIG. 5B (160 h incubation). The transfection conditions were 100 nM siRNA molecules complexed with atuFECT01 (1 μg/ml).

### beta-Arginyl-2,3-diaminopropionic acid-N-palmityl-N-oleyl-amide trihydrochloride

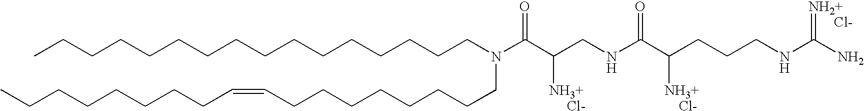

### Diphytanoylphosphatidylethanolamine

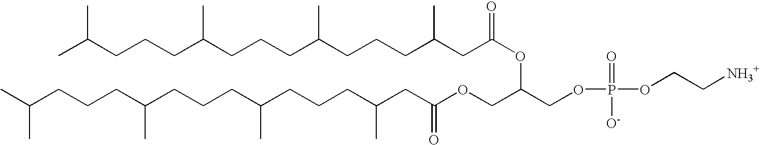

Formulation of the dsRNA Molecules

Chloroformic solutions of the cationic lipid beta-arginyl-2,3-diamino propionic acid-N-palmityl-N-oleyl-amide trihydrochloride and of the helper lipid diphythanoylphosphatidyl ethanolamine were blended such that both lipids were present in a molar ratio of 1:1. Subsequently, the chloroform was removed under vacuum and the resulting lipid film rehydrated in water at a concentration of 1 μg lipid/ml. The cationic lipids thus formed were subject to ultrasonic treatment of the dispersion for 6 minutes at room temperature in an ultrasound bath. For complexing the lipids with the dsRNA molecules the dsRNA (2 mg/l DPBS) was admixed with lipid (0.735 mg/ml ultrapure water) in a volume ratio of 1:1, vortexed and incubated at 37° C. for about 20 minutes. This formulation is also referred to herein as atuFECT01.

As maybe taken from FIGS. 5A and 5B, the various RNA molecules have a different impact on the capability on the proliferation of the PC-3 cells. The proliferation assay is a suitable assay to indicate the potency of cells to grow and thus being also an indicator of their mitogenic and apoptotic activity.

Comparing the results as depicted in FIGS. 5A and 5B it is important to note that after a growth for 72 h, the effects of a treatment of the cells using different RNA molecules is not that pronounced in contrast to the situation, where the cells were observed again after 160 h growth on 10 cm plates.

PK71AB which is a siRNA molecule, i. e. a double-stranded RNA molecule comprising 21 base pairs is directed against PKN beta (NM_{—}013355) and is thus suitable to down-regulate the PKN beta mRNA in accordance with the RNA interference mechanisms. However, PKN beta does not have any impact on the growth of PC-3 cells.

PTENAB is a double-stranded structure comprising the PTENA and PTENB sequence as depicted above. After 160 h the PC-3 cells are inhibited and the growth and survival is significantly reduced. As PC-3 cells are PTEN−/− cells, i. e. they do not have a transcriptome comprising a mRNA or other transcribed RNA coding for PTEN, this confirms the finding as disclosed in this application that an inhibition of cell proliferation and apoptosis can be induced by the use of double-stranded nucleic acids, more preferably ribonucleic acids as double-stranded ribonucleic acids which do not match to the transcriptome.

Also the use of PTENABMM shows a similar effect as described above for PTENAB. PTENABMM is a double-stranded ribonucleic acid comprising the sequences PTENAMM and PTENBMM as described above. Apart from the PC-3 cells not exhibiting a transcript of the PTEN gene, the miss match results in an even stronger response in terms of inhibited cell proliferation and thus a stronger apoptotic effect in view of the cell density when comparing the density after 72 h and 160 h, respectively.

However, using PTENA and PTENB, respectively, as single-stranded constructs, it may be taken from comparing FIGS. 5A and 5B, that the single-stranded ribonucleic acid does not result in the inhibition of PC-3 cell proliferation thus confirming the requirement of a double-stranded nucleic acid structure so as to achieve the effects as disclosed herein.

The double-stranded ribonucleic acid molecule PTENABV15 is comprised of the two strands PTENAV15 and PTENBV15 as depicted above. This molecule shows a modification pattern such the first, third and so on, i. e. any next but one nucleotide comprises a modification, in the particular case a 2′O-methylated ribose moiety, wherein the first nucleotide modified is the first nucleotide starting from the 5′ end of the antisense strand. The PTENBV15 strand, which is complementary to the PTENAV strand has the same pattern, however, there is a frame shift of one nucleotide so that the second, fourth, sixth and so on, i. e. again the next what one nucleotide is modified in the same way as the antisense strand, i. e. by a 2′O-methylated ribose.

This result confirms that the modification pattern, which is also referred to herein as one embodiment of the spatial modification pattern results in the effects described herein.

The same is also true for the double-stranded RNA molecules PTENABV10, which is also related to a PTEN mRNA or a PTEN transcript which, however, given the particular genetic background of PC-3 cells, is not present. Also this sequence results in an inhibition of the growth of PC-3 cells in the proliferation assay and is thus indicative for cell lysis and apoptosis based on the mechanism disclosed herein.

Finally, the molecule PTENABV1 consisting of the individual sequences PTENAV1 and PTENBV1 as described above, was assayed, however, is not active in the assay, i. e. this kind of molecule is not suitable to inhibit cell proliferation. Although the sequence as such is directed against PTEN and should work insofar, however, the molecule is characterised such as both strands are fully methylated at the 2′ position of the ribose, wherein such overall modification is known not to elicit a RNA interference. From this the present inventors concluded that although modification is allowable so as to trigger the effects described herein, however, the generation of a fully modified dsRNA molecule, wherein the antisense strand is fully methylated at the 2′ ribose and preferably also the sense strand, which is known not to be effective in eliciting an RNAi response, is not workable in connection with the present invention.

It is to be noted that the length of the dsRNA molecules used in this example was 21 nucleotide pairs

### EXAMPLE 5

### Mismatch Induced apoptosis

In order to confirm the finding underlying the present invention, namely that cell growth can inhibit and more particularly apoptosis induced by a double-stranded nucleic acid, preferably double-stranded ribonucleic acid which is not perfectly base pairing but exhibits at least one, preferably several mismatches compared to an optimal siRNA molecule which would elicit a RNA interference reaction, the following experiment was carried out.

HeLa cells were transfected with 100 nm siRNA using atuFECT01 (1 μg/ml), with atuFECT01 being prepared as described above in connection with example 4. The following sequences were used:

| | |
|---|---|
| PTENM | 5′- cuccuuuuguuucugcuaacg-TT |
| | 3′-TT-gaggaaaacaaagacgauugc |
| PTENMM | 5′- cucauuuucuuugugcucacg-TT |
| | 3′-TT-gaguaaaagaaacacgagugc |
| p110αM | 5′- cuccaaagccucuugcucagu-TT |
| | 3′-TT-gagguuucggagaacgaguca |
| p110αMM | 5′- cugcaaacccuguugcucacu-TT |
| | 3′-TT-gacguuugggacaacgaguga |

Underlined nt = corrresponding to the introduced mismatches
Capital = desoxy nt

The thus treated cells were lysed after 48 and 72 h, respectively, and analysed by immunoblot using the indicated antibodies.

Please note that in the above sequences, the miss matches of the dsRNA molecules are indicated by underlining. Therefore, the miss matches in case of PTENMM were based on positions 4, 9, 13 and 18 on the antisense strand with nucleotide no. 1 being at the 5′ end, wherein such miss matches referred to the PTEN mRNA as present in HeLa (as the transkriptome of HeLa still comprises PTEN mRNAs).

In case of p110α which is a subunit of P13K as described in example 3, the miss matches were at positions 3, 8, 12 and 20 on the sense strand with a first nucleotide being the nucleotide at the 5′ end and the corresponding positions on the sense strand. In any case, the double-stranded RNA molecules comprised a first stretch of 21 nucleotides and a second stretch of 21 nucleotides. In each case, there was a didesoxy nucleotide, namely TT at the 3′ end of each of the sense and the antisense strand.

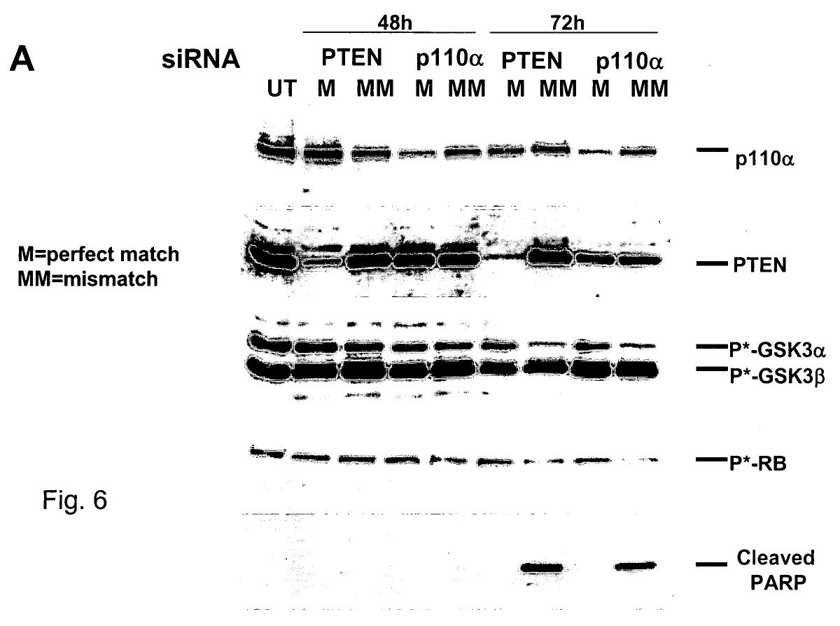

The results are depicted in FIG. 6. As may be taken from FIG. 6, the dsRNA molecule directed against p110α results in a decrease of the p110α protein. In case the double-stranded RNA molecule was perfectly matching to the target mRNA, i. e. the p110α mRNA. The same is true for PTEN.

A further read-out was RB and GSK3α which, in their dephosphorylated form are indicative of inhibition of proliferation, wherein in both cases dephosphorylation means activation. A further read-out is the cleaved PARP signal which indicates apoptosis. It is well established that activated caspase-3 cleaves the death substrate PARP (Lazebnik et al., 1994, Nature 371, 346-347).

Again referring to FIG. 6, at least after 72 h, the PTENMM and p110αMM obviously reduce the extent to which RB and GSK3α are phosphorylated. Additionally, for the very same dsRNA molecules it can be established that cleaved part is present at a significant level indicating that the cells treated with these dsRNA molecules undergo apoptosis. This confirms that even if a target sequence is present, the use of a not perfectly matching double-stranded RNA molecule results in a stress reaction, including, but not limited to, apoptosis, and results finally in growth inhibition and/or cell arrest and/or cell lysis.

### EXAMPLE 6

### dsRNA Based Therapy in a Human PC3 Mouse Model

Experimental Design

The in vivo experiments were conducted corresponding the Good Laboratory Practice for Nonclinical Laboratory Studies (GLP Regulations) of the Food and Drug Administration (U.S. Department of Health and Human Services, Food and Drug Administration, Rockville, Md.) and in accordance with the german animal protection law as legal basis Bundesgesetzblatt, I, May 25, 1998, p. 1094. The permission of the regional governmental authority was present (Landesamt für Arbeitsschutz, Gesundheitsschutz und technische Sicherheit Berlin). Human prostate carcinoma cells PC-3 (American Type Culture Collection (ATCC) 2002, Manassas, Va. 20108) were grown in F-12K Kaighn's modification medium (Gibco BRL) containing 2 mM Glutamine (Gibco BRL), 20 mM Hepes (Biochrom) and 10% fetal bovine serum (Gibco BRL). Cultivated cells were trypsinated and harvested following stopping the trypsin effect by medium. Washing procedures (PBS; Centrifugation 5 min/1.000 rpm) are added and, finally, the pellet is resuspended considering the cell number and volume to be inoculated.

Animals and Cell Challenge

Male Shoe: NMRI-nu/nu mice (DIMED Schönwalde GmbH) maintained under SPF conditions (Laminar air flow equipment, Scantainer, Scanbur) served as recipients for PC3 cells. The animals, aged 8 weeks and weighing 28-32 g, were inoculated subcutaneously 5×10⁶/0.1 ml tumor cells into the left inguinal region. 17 days post cell challenge the animals were randomised according to the 4 treatment groups consisting of 6 animals per group each. The tumor sizes were ranging between 150 and 160 mm³ per treatment group. The animals were inspected successively inclusive of protocolling the findings. Ssniff NM-Z, 10 mm, autoclavable (ssniff Spezialdiäten GmbH) was administered as fortified diet, tap water for drinking was acidified, both ad libitum.

Formulations

The formulations were prepared as described above in connection with example 4.

Additionally, 300 μl of this formulation were injected i. v. into a 30 g mouse which results in a dosage of 10 mg siRNA and 3.7 mg lipid per kg body weight, respectively. The naked siRNA (2 mg/ml DPBS) was filled up in the same volume ratio with DPBS and treated adequately. The complexes were freshly prepared for each treatment.

siRNA Treatments

The siRNA preparations including PBS as control were administered intravenously via tail vene. The treatment schedule consisted of 9 consecutive daily injections. The dose level amounted 10 mg siRNA respectively 3.7 mg lipid per kg body weight performing an injection volume of 0.3 ml/30 g mouse. The following treatment groups were included:
PBS;
PTEN/AB/Naked;
PTEN/AB
   Evaluations

Body weights were registered regularly for the experimental duration to evaluate the physical constitution of the animals. The tumors were measured in two dimensions by means of a pair of callipers, daily under the treatment period and later 3 times a week. The volume was calculated according to V (mm³) =ab²/2 with b<a (In vivo cancer models. 1976-1982. Washington, D.C.: National Cancer Institute 1984 (NIH Publication No. 84-2635, February 1984)). In general, the cell number performed for the approaches causes a 100% tumor take.

Blood parameters were evaluated by means of blood punctures from the orbital sinus:

Enzymes: ALT, AST, AP;

Cellular constituents: WBC, RBC, PLT, Hb, Hkt;

Leukocyte differentiation.

For histological analysis samples of tumor and liver tissues were fixed in 5% formaldehyde and paraffin embedded. Routinely, the sections were HE stained.

The results concerning tumor sizes were statistically verified by the u-test of Mann and Whitney.
/atuFect01;
MTP18/atuFect01.
PTENV1/atuFect01
PTENGB/atuFecto1

The following double-stranded ribonucleic acid and antisense molecules were used:

| | |
|---|---|
| PTENA | 5′cuccuuuuguuucugcuaacg-TT |
| PTENB | 5′cguuagcagaaacaaaaggag-TT |
| MTP18A | 5′ugccuucuugccuuugucuau |
| MTP18B | 5′auagacaaaggcaagaaggca |
| PTENAV1 | 5′cuccuuuuguuucugcuaacg |
| PTENBV1 | 5′cguuagcagaaacaaaaggag |
| PTEN53 GB3.5 | 5′cuccuu [symbol] Gcuaacg |

Please note that any 2′O-methyl modified ribonucleotide is indicated in bolt and underlined, capital letters indicate desoxyribonucleotides and any letters in capital and italic are indicative of desoxyribonucleotides comprising a phosphothioate.

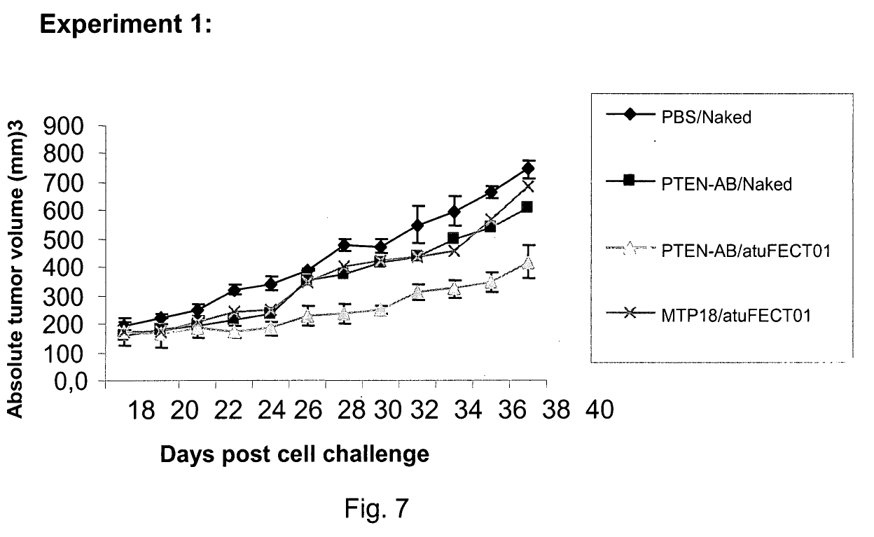

The results are depicted in FIG. 7.

It may be taken from FIG. 7 that after day 20 the response to PBS compared to the response triggered by the administration of PTENAB/atuFECT01 is statistically significant according to Mann and Whitney (P=0.05) and the absolute tumor volume increased less compared to any other treatment regimen. The administration of the various formulations occurred on day 17 to 18 daily.

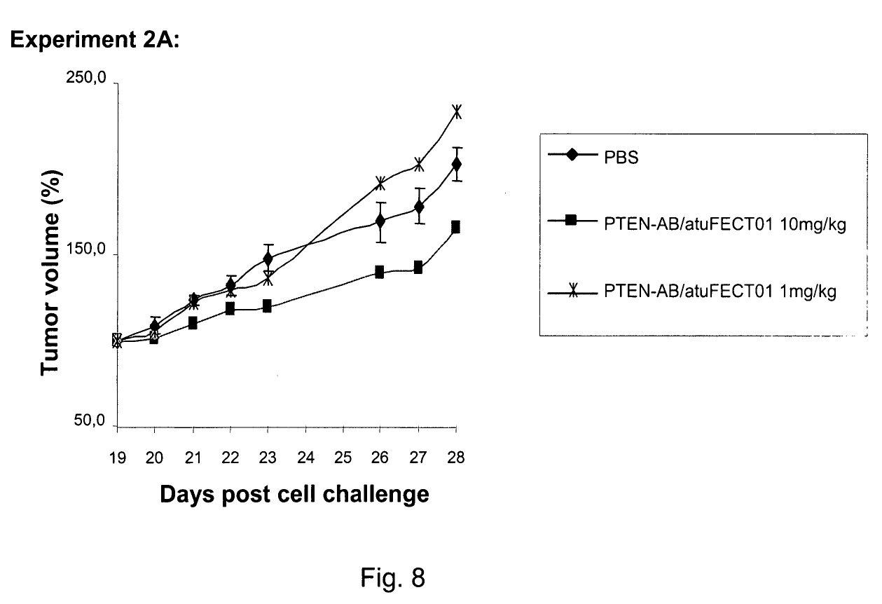

In a further experiment the animals were challenged with PBS and alternatively with PTENAB/atuFECT01 10 mg/kg and PTENAB/atuFECT01 1 mg/kg. The respective formulations were administered daily on days 19 to 26. The result is depicted in FIG. 8.

As may be taken from FIG. 8 the relative tumor volume increases less if a bigger amount of the formulation is administered thus confirming the dose-dependent reaction observed also in in vitro studies.

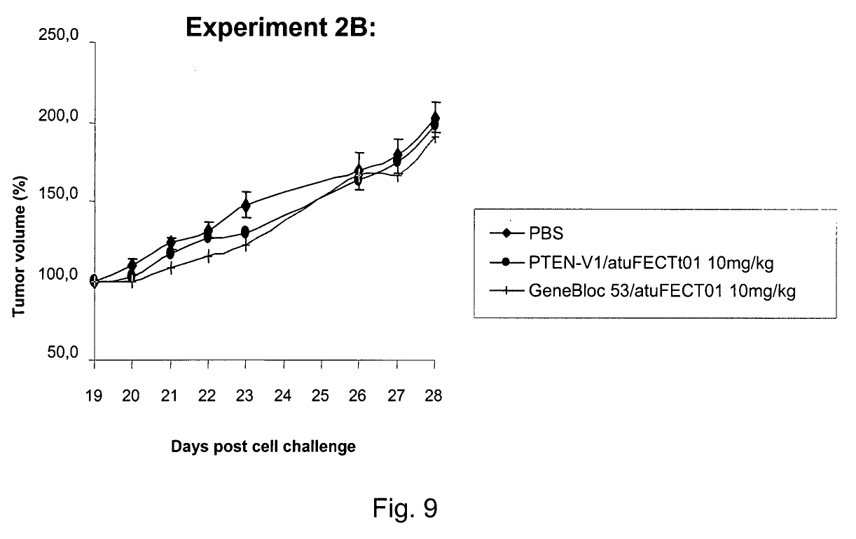

In a further experiment, the result of which is depicted in FIG. 9, the impact of PBS, of PTEN-V1/atuFECT01 10 mg/kg and of Gene Bloc 53/atuFECT01 10 mg/kg was assessed in the very same animal model.

It may be taken from this diagram that using the PTEN-V1 construct which consists of the PTENAV1 and PTENBV1 strands as depicted above was formulated as described above with the particular lipids, wherein such molecule was fully modified on both the sense strand and antisense strand such that any 2′OH position was modified to be a 2′O-methyl. This molecule was actually not effective in the mouse model and the tumor volume increased was similar to the one upon treatment with PBS. The reason for this is that the full modification, preferably the full modification of the antisense strand, made the strand inactive so that the effects observed when this kind of double-stranded RNA molecule is used in a cellular system, where no target nucleic acid, i. e. mRNA, more precisely PTEN mRNA is present, would result in a stress response as described above.

Also Gene Bloc 53/atuFECT01 which is directed against PTEN is not effective in reducing the tumor volume which is not surprising given the fact that the particular PC-3 cells do not provide for a target and, in accordance with the experiments described in example 4, a single-stranded oligonucleotide is not suitable to induce the effects observed and disclosed herein.

The features of the present invention disclosed in the specification, the claims and/or the drawings may both separately and in any combination thereof be material for realizing the invention in various forms thereof.

1. A pharmaceutical composition comprising a nucleic acid, wherein the nucleic acid comprises a double-stranded structure and the double-stranded structure comprises a first strand and a second strand,
wherein the first strand comprises a first stretch of contiguous nucleotides and the second strand comprises a second stretch of contiguous nucleotides,
wherein the first stretch is not complementary to a target nucleic acid of a cell of an organism to be treated with said medicament and/or
wherein the second stretch is different from a target nucleic acid of a cell of an organism to be treated with said medicament;
and a pharmaceutically acceptable carrier.

2. The composition according to claim 1, wherein said nucleic acid is a ribonucleic acid.

3. The pharmaceutical composition according to claim 1, wherein said target nucleic acid is any nucleic acid of a cell of an organism to be treated using said pharmaceutical composition.

4. The composition according to claim 3, wherein said target nucleic acid in said cell is an mRNA.

5. The composition according to claim 1, wherein said target nucleic acid is an element of the transcriptome of a cell of an organism to be treated with said pharmaceutical composition.

6. The composition according to claim 5, wherein said target nucleic acid is, all elements of the transcriptome of said organism.

7. The composition according to claim 1, wherein said cell is a pathological cell which is associated with the disease which is to be treated and/or prevented by the pharmaceutical composition.

8. The composition according to claim 1, further comprising at least one lipid.

9. The composition according to claim 8, wherein said lipid is a cationic lipid.

10. The composition according to claim 9, wherein said lipid is beta-arginyl-2,3-diaminopropionic acid-N-palmityl-N-oleyl-amide trihydrochloride.

11. The composition according to claim 8, further comprising a helper lipid.

12. The composition according to claim 11, wherein said helper lipid is diphytanoylphosphatidylethanolamine.

13. A method for the treatment of a disease in a subject, comprising administering to a subject suffering from said disease an effective amount of a composition according to claim 1, wherein said cell of an organism is a cell of said subject.

14. The method according to claim 13, wherein said disease is cancer.

15. A method of treating a disease in a subject, comprising administering to a subject suffering from said disease an effective amount of a nucleic acid, wherein said nucleic acid comprises a double-stranded structure and the double-stranded structure comprises a first and a second strand,
wherein said first strand comprises a first stretch of contiguous nucleotides and the second strand comprises a second stretch of contiguous nucleotides,
wherein said first stretch is not complementary to a target nucleic acid of a cell of said subject to be treated with said nucleic acid and/or
wherein said second stretch is different from a target nucleic acid of a cell of said subject to be treated with said nucleic acid.

16. The method according to claim 15, wherein said nucleic acid is a ribonucleic acid.

17. The method according to claim 15, wherein said target nucleic acid is an RNA

18. The method according to claim 17, wherein said target nucleic acid is any or all elements of the transcriptome of a cell of an organism.

19. The method according to claim 15, wherein said cell of said subject is a pathological cell involved in said disease.

20. The method according to claim 15, wherein said first stretch is complementary to a nucleic acid of a non-pathological cell of said subject and/or
wherein the second stretch is identical to a target nucleic acid of a non-pathological cell of said subject.

21. The method according to claim 20, wherein said nucleic acid is an mRNA.

22. The method according to claim 21, wherein the target nucleic acid of the non-pathological cell differs from the target nucleic acid of the pathological cell at one or more nucleotide positions.

23. The method according to claim 15, wherein said disease is a tumor or cancer.

24. The method according to claim 19, wherein said pathological cell is tumor suppressor-defective and said first stretch of contiguous nucleotides is complementary to the nucleic acid coding for the functional tumor suppressor and/or
said second stretch of contiguous nucleotides is identical to the nucleic acid coding for the functional tumor suppressor.

25. The method according to claim 24, wherein said first stretch is complementary to the nucleic acid coding for the functional tumor suppressor for which the pathological cell is tumor suppressor-defective, and
the second stretch is identical to the nucleic acid coding for the functional tumor suppressor for which the pathological cell is tumor suppressor-defective.

26. The method according to claim 19, wherein the pathological cell is lacking a gene of the functional tumor suppressor or a transcript thereof.

27. The method according to claim 19, wherein the pathological cell is lacking a gene or a transcript thereof providing for a functionally active tumor suppressor

28. The method according to claim 27, wherein the gene or the transcript thereof comprises one or several mutations, wherein each mutation is selected from the group consisting of point mutations and deletion mutations, wherein said mutation(s) results in a functionally inactive tumor suppressor.

29. The method according to claim 15, wherein said nucleic acid is RNA interference response-negative.

30. The method according to claim 29, wherein said nucleic acid is RNA interference response-negative in pathological cells of the subject.

31. The method according to claim 29, wherein said nucleic acid is RNA interference response-positive in non-pathological cells of said subject.

32. The method according to claim 15, wherein the nucleic acid induces a stress response in said cells.

33. The method according to claim 32, wherein said stress response is apoptosis and/or inhibition of proliferation of the pathological cells.


SEQUENCE LISTING (34 sequences)

SEQ ID NO: 1 - RNA, 56 nt, Artificial Sequence
  Description of Artificial Sequence Synthetic oligonucleotide
guucacuguaaagcuggaaagggaaaaaaaaaaaacccuuuccagcuuuacaguga

SEQ ID NO: 2 - RNA, 56 nt, Artificial Sequence
  Description of Artificial Sequence Synthetic oligonucleotide
guucacucuaaaggugcaaacggaaaaaaaaaaaaccguuugcaccuuuagaguga

SEQ ID NO: 3 - DNA, 23 bp, Artificial Sequence
  Description of Combined DNA/RNA Molecule Synthetic oligonucleotide
cuccuuuuguuucugcuaacgtt

SEQ ID NO: 4 - DNA, 23 bp, Artificial Sequence
  Description of Combined DNA/RNA Molecule Synthetic oligonucleotide
cguuagcagaaacaaaaggagtt

SEQ ID NO: 5 - DNA, 23 bp, Artificial Sequence
  Description of Combined DNA/RNA Molecule Synthetic oligonucleotide
cucauuuucuuugugcucacgtt

SEQ ID NO: 6 - DNA, 23 bp, Artificial Sequence
  Description of Combined DNA/RNA Molecule Synthetic oligonucleotide
cgugagcacaaagaaaaugagtt

SEQ ID NO: 7 - DNA, 23 bp, Artificial Sequence
  Description of Combined DNA/RNA Molecule Synthetic oligonucleotide
cuucucgcaguacaggcucuctt

SEQ ID NO: 8 - DNA, 23 bp, Artificial Sequence
  Description of Artificial Sequence Synthetic oligonucleotide
gagagccuguacugcgagaagtt

SEQ ID NO: 9 - RNA, 21 nt, Artificial Sequence
  Description of Artificial Sequence Synthetic oligonucleotide
cuccuuuuguuucugcuaacg

SEQ ID NO: 10 - RNA, 21 nt, Artificial Sequence
  Description of Artificial Sequence Synthetic oligonucleotide
cguuagcagaaacaaaaggag

SEQ ID NO: 11 - RNA, 21 nt, Artificial Sequence
  Description of Artificial Sequence Synthetic oligonucleotide
cuccuuuuguuucugcuaacg

SEQ ID NO: 12 - RNA, 21 nt, Artificial Sequence
  Description of Artificial Sequence Synthetic oligonucleotide
cguuagcagaaacaaaaggag

SEQ ID NO: 13 - RNA, 19 nt, Artificial Sequence
  Description of Artificial Sequence Synthetic oligonucleotide
uaaguucuagcuguggugg

SEQ ID NO: 14 - RNA, 19 nt, Artificial Sequence
  Description of Combined DNA/RNA Molecule Synthetic oligonucleotide
ccaccacagcuagaacuua

SEQ ID NO: 15 - DNA, 23 bp, Artificial Sequence
  Description of Artificial Sequence Synthetic oligonucleotide
cuccuuuuguuucugcuaacgtt

SEQ ID NO: 16 - DNA, 23 bp, Artificial Sequence
  Description of Artificial Sequence Synthetic oligonucleotide
cguuagcagaaacaaaaggagtt

SEQ ID NO: 17 - DNA, 23 bp, Artificial Sequence
  Description of Combined DNA/RNA Molecule Synthetic oligonucleotide
cucauuuucuuugugcucacgtt

SEQ ID NO: 18 - DNA, 23 bp, Artificial Sequence
  Description of Combined DNA/RNA Molecule Synthetic oligonucleotide
cgugagcacaaagaaaaugagtt

SEQ ID NO: 19 - DNA, 23 bp, Artificial Sequence
  Description of Combined DNA/RNA Molecule Synthetic oligonucleotide
cuccaaagccucuugcucagutt

SEQ ID NO: 20 - DNA, 23 bp, Artificial Sequence
  Description of Combined DNA/RNA Molecule Synthetic oligonucleotide
acugagcaagaggcuuuggagtt

SEQ ID NO: 21 - DNA, 23 bp, Artificial Sequence
  Description of Combined DNA/RNA Molecule Synthetic oligonucleotide
cugcaaacccuguugcucacutt

SEQ ID NO: 22 - DNA, 23 bp, Artificial Sequence
  Description of Artificial Sequence Synthetic oligonucleotide
agugagcaacaggguuugcagtt

SEQ ID NO: 23 - RNA, 21 nt, Artificial Sequence
  Description of Artificial Sequence Synthetic oligonucleotide
ugccuucuugccuuugucuau

SEQ ID NO: 24 - RNA, 21 nt, Artificial Sequence
  Description of Artificial Sequence Synthetic oligonucleotide
auagacaaaggcaagaaggca

SEQ ID NO: 25 - DNA, 21 bp, Artificial Sequence
  Description of Combined DNA/RNA Molecule Synthetic oligonucleotide
cuccuuttgtttctgcuaacg

SEQ ID NO: 26 - DNA, 23 bp, Artificial Sequence
  Description of Combined DNA/RNA Molecule Synthetic oligonucleotide
agaccacaaacugaggauugctt

SEQ ID NO: 27 - DNA, 23 bp, Artificial Sequence
  Description of Combined DNA/RNA Molecule Synthetic oligonucleotide
gcaauccucaguuuguggucutt

SEQ ID NO: 28 - DNA, 23 bp, Artificial Sequence
  Description of Combined DNA/RNA Molecule Synthetic oligonucleotide
agacgacaaagucagcauugctt

SEQ ID NO: 29 - DNA, 23 bp, Artificial Sequence
  Description of Combined DNA/RNA Molecule Synthetic oligonucleotide
gcaaugcugacuuugucgucutt

SEQ ID NO: 30 - DNA, 23 bp, Artificial Sequence
  Description of Combined DNA/RNA Molecule Synthetic oligonucleotide
augccacuucaguuggugacatt

SEQ ID NO: 31 - DNA, 23 bp, Artificial Sequence
  Description of Combined DNA/RNA Molecule Synthetic oligonucleotide
ugucaccaacugaaguggcuatt

SEQ ID NO: 32 - DNA, 23 bp, Artificial Sequence
  Description of Combined DNA/RNA Molecule Synthetic oligonucleotide
uagcgaguucacuugcugacatt

SEQ ID NO: 33 - DNA, 23 bp, Artificial Sequence
  Description of Combined DNA/RNA Molecule Synthetic oligonucleotide
ugucagcaagugaacucgcuatt

SEQ ID NO: 34 - DNA, 21 bp, Artificial Sequence
  Description of Combined DNA/RNA Molecule Synthetic oligonucleotide
cucauuttctttgtgcucacg